(19) United States
(12) Patent Application Publication
(10) Pub. No.: US 2023/0310332 A1
(43) Pub. Date: Oct. 5, 2023

(54) METHOD FOR MANUFACTURING A MULTI-LAYERED FILM STRUCTURE AND METHOD FOR MANUFACTURING MULTI-LAYERED MICROSTRUCURES

(71) Applicant: MLMC THERAPEUTICS APS, Copenhagen S (DK)

(72) Inventors: Ritika Singh Petersen, Herlev (DK); Benjamin Robotham, Valby (DK); David Williamson, Frederiksberg (DK)

(73) Assignee: MLMC THERAPEUTICS APS, Copenhagen S (DK)

(21) Appl. No.: 18/011,867

(22) PCT Filed: Jun. 22, 2021
(86) PCT No.: PCT/DK2021/050199

§ 371 Date: Dec. 21, 2022

(30) Foreign Application Priority Data
Jun. 25, 2020 (DK) PA202070417

(51) Int. Cl. A61K 9/50

(52) U.S. Cl. CPC A61K 9/5084; A61K 9/5089

(57) ABSTRACT

There is provided a method of manufacturing a multi-layered film structure on a handling substrate. The film structure may hold a core film layer, which may hold an active ingredient. There is also provided method for manufacturing multi-layered microstructures. The microstructures may be manufactured based on a provided multi-layered film structure having a core film layer holding an active ingredient. The active ingredient may be a drug, and the microstructures may be used for drug delivery.

### TECHNICAL FIELD

The aspects of the disclosed embodiments relate to a method of manufacturing of multi-layered film structure on a handling substrate. The film structure may hold a core film layer, which may hold an active ingredient.

The aspects of the disclosed embodiments further relate to a method for manufacturing multi-layered microstructures. The microstructures may be manufactured based on a provided multi-layered film structure having a core film layer holding an active ingredient. The active ingredient may be a drug, and the microstructures may be used for drug delivery.

### BACKGROUND

Drug forms consisting of several layers, for example laminated or multilayer tablets, are increasingly being used, for example in order to combine active ingredients which are incompatible with one another or to bring about release of initial and maintenance doses in the case of controlled-release drug forms.

US patent application No. US 2016/0206513 discloses a method for fabrication of multi-layered micro-containers for drug delivery, which method may be used for manufacturing of drug loaded micro-containers on wafer scale. A method is disclosed which comprises the steps of: a) preparing a multi-layered film comprising at least a core layer and a barrier layer, wherein the core layer comprises at least an active ingredient; and b) subjecting the multi-layered film to a hot embossing step using an embossing stamp having protrusions that allows for generation of the one or more micro-container(s) containing the active ingredient, such that the barrier layer partially encloses the core layer. The multi-layered film is obtained by using a 4-inch single crystal silicon wafer as a handling substrate, where a polymer-drug core layer is fabricated by spin coating of a solution of polycaprolactone (PCL) and the diuretic drug furosemide on the silicon wafer. The resulting film thickness of the core layer was 15 μm. A polymer barrier layer was deposited onto the polymer-drug core layer by spin coating of a solution of PCL. The resulting thickness of the barrier layer was 10 μm.

However, spin-coating of the multiple layers is not very well suited for large scale production of micro-containers or microstructures holding an active ingredient, which large scale production may require a roll-to-roll scale production, with the film layers being manufactured on a roll of a flexible material.

Thus, there is a need for an improved method of coating a substrate with several thin film layers, with each layer having a uniform thickness, which method is suited for large scale production of micro-containers or microstructures, where the micro-containers or microstructures may hold an active ingredient in form of a drug to be released.

### SUMMARY

It is an object of the aspects of the disclosed embodiments to provide a solution for providing a substrate with thin film layers having uniform thickness.

This object is achieved in accordance with a first aspect by providing a method for manufacturing a multi-layered film structure, said method comprising the steps of:
a) providing a handling substrate having a length and a width, which handling substrate is of a deformable material having a deformable top surface, or which handling substrate holds a deformable top surface layer;
b) providing a plurality of film layers on top of the deformable surface of the handling substrate; wherein
   one or more of the film layers are provided by a slot die coating process, said slot die coating process of a film layer comprising:
   supplying one or more materials for forming the film layer to a slot die head; and
   dispensing the supplied materials in a liquid form from the slot die head while moving the slot die head relative to the handling substrate, said dispensed liquid form material being deposited as a wet film layer on top of the handling substrate, or on top of the deformable top surface layer, or on top of one or more previously provided film layers.

It is also within an object of the present disclosure to provide a solution for manufacturing a number of microstructures holding one or more film layers provided on the substrate.

Thus, in a possible implementation form of the first aspect, the method further comprises:
c) subjecting the plurality of film layers to a punching step for generation of a plurality of microstructures, said punching being performed by use of a rigid stamp having a plurality of protrusions defining a plurality of cavities, wherein the depth of the cavities is larger than the thickness of the plurality of film layers, and lower than the combined thickness of the plurality of film layers and the deformable surface part of the handling substrate, thereby allowing the protrusions of the rigid stamp to penetrate all the way through the plurality of film layers during punching to divide the plurality of film layers into a plurality of separated microstructures.

In an implementation form of the first aspect, there is therefore provided a method for manufacturing a plurality of microstructures, said method comprising the steps of:
a) providing a handling substrate having a length and a width, which handling substrate is of a deformable material having a deformable top surface, or which handling substrate holds a deformable top surface layer;
b) providing one or more film layers on top of the deformable surface of the handling substrate; and
c) subjecting the one or more film layers to a punching step for generation of the plurality of microstructures, said punching being performed by use of a rigid stamp having a plurality of protrusions defining a plurality of cavities, wherein the depth of the cavities is larger than the thickness of the one or more film layers, and lower than the combined thickness of the one or more film layers and the deformable surface part of the handling substrate, thereby allowing the protrusions of the rigid stamp to penetrate all the way through the one or more film layers during punching to divide the one or more film layers into a plurality of separated microstructures; wherein
   one or more of the film layers are provided by a slot die coating process, said slot die coating process of a film layer comprising:
   supplying one or more materials for forming the film layer to a slot die head; and
   dispensing the supplied materials in a liquid form from the slot die head while moving the slot die head relative to the handling substrate, said dispensed liquid form material being deposited as a wet film layer on top of the handling substrate, or on top of the deformable top surface layer, or on top of one or more previously provided film layers.

In a possible implementation form of the first aspect, a plurality of film layers are provided on top of each other by a plurality of said slot die coating processes subsequently following each other.

In a possible implementation form of the first aspect, at least one or all of said slot die coating processes further comprises heating or drying of the deposited wet film layer to obtain a solid film layer.

In a possible implementation form, the wet film layer is dried or heated by air flow drying or heated by infrared heating. Drying or heating may be performed to evaporate solvents and change properties of the wet film layer.

In a possible implementation form, the wet film layer is exposed to ultra-violet light, which may be used to obtain crosslink photoinitiated polymerization.

In a possible implementation form of the first aspect, at least one of said slot die coating processes further comprises application of pressure to the obtained solid film layer, such as exposing the solid film layer together with the handling substrate and any previously provided film layers to a calendaring process.

By using a calendaring process, the one more solid film layers are compressed, and the porosity may be reduced. It should be understood that the drying and/or heating of the wet film layer and the application of pressure to the solid film layer should be performed before subjecting the film layers to a punching process.

In a possible implementation form of the first aspect, then for at least one of said slot die coating processes, at least part of the material(s) for forming the film layer is/are supplied to the slot die head in solid form, and the slot die head is heated to a temperature for melting the supplied solid material to thereby enable dispensing of the supplied materials in a liquid form from the slot die head.

In a possible implementation form of the first aspect, then for at least one of said slot die coating processes, at least part of the material(s) for forming the film layer is/are supplied to the slot die head in liquid form.

In a possible implementation form of the first aspect, then for at least one of said slot die coating processes, the slot die head is supplied with at least one material being pre-heated above its melting point and being fed to the slot die head in liquid form. Here, the slot die head may be heated to a temperature for maintaining said at least one material in liquid form.

The material may be supplied as a liquid solution holding a polymer in a solvent, as a slurry holding an active ingredient, or as a solid material, which is melted before entering the slot die head, or entering the slot die head in solid form to be melted within the slot die head.

The supplied solid materials could for example be thermoplastics or thermosets, such as crosslinked hydrogels or 3D printing resins.

The supplied materials may be one or more materials selected from the list comprising: polycaprolactone, (PCL), polylactic acid (PLA), polyglycolic acid (PGA), hydroxypropylmethyl cellulose (HPMC), polymethacrylate (PMMA), Eudragits (Poly(methacylic acid-co-methyl methacrylate), ethyl cellulose (EC), polyvinyl alcohol (PVA), polyvinylpyrollidone (PVP), polyethylene glycol (PEG), polyethylene glycol methacrylate (PEGMA), polyethylene glycol dimethacrylate (PEGDMA), polylactic-co-glycolic acid) (PGLA), polyacrylic acid (PAA).

The supplied materials may also comprise one or more co-polymers of at least one of the above-mentioned list of polymers.

The supplied solid materials could also comprise or include materials, which turn liquid when slightly heated and become solid at room temperature like: wax, oils, paraffins, butter and other oil-based, fat-based materials, hydrogels.

In a possible implementation form of the first aspect, then for at least one of said slot die coating processes of a film layer, the slot die head is supplied with at least two different materials being fed from at least two different outlets in liquid form. Here, the slot die head may be heated to a temperature for maintaining the received materials in liquid form.

As an example, a polymer, such as PCL and a small molecule drug containing solution holding a drug, such as furosemide, can be fed from two different outlets into the slot die head, which is heated to 80-100 degrees C., where the PCL remains melted and gets blended with the drug solution. This blend is then coated on a substrate while blending continues within the slot die head itself.

In a possible implementation form of the first aspect, the handling substrate may hold a deformable top surface layer, said deformable top surface layer being provided by a slot die coating process.

In a possible implementation form of the first aspect, the film layer(s) comprises/comprise a core layer holding an active ingredient.

The core layer may comprise an active ingredient in the form of a drug or in the form of a drug matrix, which drug matrix may be made of at least one polymer and an active ingredient. Examples of active ingredients: small organic drug molecules, proteins, peptides, vitamins, minerals antibodies, antibody fragments, vaccines, RNA, DNA, antibiotics or combinations, screening materials, diagnostic materials like certain metals, enzymes. The thickness of the core layer may be a function of the active ingredient. Thus, the thickness of the core layer may be proportional to a desired release period of the active ingredient from the core layer of the microstructure.

In a possible implementation form of the first aspect, then for at least one of said slot die coating processes of a film layer, the slot die head is supplied with a blend of a biodegradable polymer and a solid material, which can sublime, or which have a lower boiling point than the polymer, said blend being dissolved in an solvent for being dispensed from the slot die head and deposited as a wet film layer, wherein the deposited wet film layer is heated for sublimation or for evaporation of the solid material.

In a possible implementation form of the first aspect, the step of providing a plurality of film layers or providing one or more film layers on top of the deformable surface of the handling substrate comprises:
providing several film layers on top of the deformable surface of the handling substrate, said film layers comprising at least the following sequence of layers on top of the deformable surface of the handling substrate:
   i) a lid film layer;
   ii) a core film layer; and
   iii) a shell film layer.

In a possible implementation form of the first aspect, one or more additional functional film layers, such as one or more mucoadhesive layers may be provided by one of said slot die coating processes.

In a possible implementation form of the first aspect, each of said provided film layers are provided by one of said slot die coating processes.

In a possible implementation form of the first aspect, the shell film layer has a greater thickness than the lid film layer, such as a thickness being at least 1.5 times the thickness of the lid film layer, such as a least 2 times the thickness of the lid film layer, such as a least 5 times the thickness of the lid film layer, such as a least 10 times the thickness of the lid film layer.

In a possible implementation form of the first aspect, the core film layer has a greater thickness than the lid film layer, such as a thickness being at least 1.5 times the thickness of the lid film layer, such as a least 2 times the thickness of the lid film layer, such as a least 5 times the thickness of the lid film layer, such as a least 10 times the thickness of the lid film layer.

For oral drug delivery applications, the shell film layer may be a protective layer for the core layer, which may hold an active ingredient, and the shell film layer may be made of a material selected from the list: biodegradable and biocompatible thermoplastics, thermo sets like hydrogels, waxes, paraffins, collagen, Ph sensitive polymers like Eudragit, mucoadhesive polymers like chitosan.

The material used for the lid film layer may be selected from the same list as for the shell film layer. The shell and lid film layers can be same of the same material with different thicknesses so that they degrade at different times in the human system. Therefore, precise control in the uniformity and thickness of such films is of great importance, which precise control can be obtained by slot die coating of the film layers.

In a possible implementation form of the first aspect, the lid film layer may be provided on top of the deformable surface of the handling substrate by supplying a solution of a solvent holding a bio-compatible or biodegradable polymer, which is dispensed from the slot die head and deposited as a wet film layer, followed by a heating process for drying the wet film layer. Here, the solution may for example hold a polymer such as polycaprolactone, PCL, or such polylactic acid, PLA, which may be dissolved in a solvent, such as chloroform or dichloromethane, DCM. The wet film layer may be heated to at least 40 or 50 degrees C. for drying of the wet film layer by evaporation of the solvent.

In a possible implementation form of the first aspect, the lid film layer may be provided on top of the deformable surface of the handling substrate by dispensing a bio-compatible or biodegradable polymer from the slot die head in melted form and depositing the polymer as a film layer of melted polymer. The slot die head may be heated to a temperature for maintaining polymer in melted form. Here, the polymer may for example be polycaprolactone, PCL, or such polylactic Acid, PLA, and the slot die head should be heated to a temperature of at least 80 degrees C. A cooling or drying process may follow the deposition of the melted film layer.

In a possible implementation form of the first aspect, the lid film layer may be provided by supplying the slot die head with a blend of a biodegradable polymer and a solid material, which can sublime, or which have a lower boiling point than the polymer, said blend being dissolved in an solvent for being dispensed from the slot die head and deposited as a wet film layer. The deposited wet film layer may be heated for sublimation or for evaporation of the solid material. In a possible implementation form of the first aspect, the sublimation or evaporation of the solid material from the deposited wet film layer is followed by an oxygen plasma treatment. As an example: Polycaprolactone (PCL), or polylactic Acid (PLA), and wax or camphor wax both can be dissolved in an organic solvent, such as chloroform or dichloromethane (DCM). Thus, the solvent may be dispensed from slot die head at room temperature. The deposited wet film layer may be heated to about 40-50 degrees C. for sublimation or evaporation. The dried polymer layer may be oxygen plasma treated for a short period, such as no longer than 1 min, such as about 30 sec. The oxygen plasma treatment may make the polymer layer hydrophilic for a following deposited film layer, which may hold an active ingredient.

In a possible implementation form of the first aspect, the core film layer may be provided on top of the lid layer by supplying the slot die head with a mix of two solutions, wherein the first solution may hold a water soluble polymer, such as poly acrylic acid (PAA), and the second solution may hold the active ingredient, such as a drug, wherein the mix is dispensed from the slot die head and deposited as a wet film layer, and wherein the deposited wet film layer may be heated to be dried. The first solution may be a solution of water and a water-soluble polymer, such as poly acrylic acid (PAA), and the second solution may be a solution of a drug, such as furosemide, in Acetone. The mix of solutions may be dispensed from the slot die head at room temperature. The deposited wet film layer may be heated to at least 50 degrees C. for about 1 hour.

In a possible implementation form of the first aspect, the core film layer may be provided on top of the lid layer by supplying the slot die head with a slurry holding the active ingredient, such as a drug, wherein the slurry is dispensed from the slot die head and deposited as a wet film layer, where after the deposited wet film layer is dried or heated to be dried. The active ingredient may be furosemide, which may be suspended in acetone to obtain the slurry. The wet film may be dried at about 40 degrees C. for about 1 min.

In a possible implementation form of the first aspect, the core film layer may be provided on top of the lid layer by supplying the slot die head with a mix of two solutions, wherein the first solution may hold a hydrophobic polymer and the second solution may hold an active ingredient, such as a drug, wherein the mix is dispensed from the slot die head and deposited as a wet film layer, and wherein the deposited wet film layer may be heated for changing the crystallinity of the deposited film layer. The mix of solutions may be dispensed from slot die head at room temperature. The deposited wet film layer may be heated to at least 80 degrees C. for about 15-30 sec. The first solution may be a solution of a hydrophobic polymer, such as PCL, and chloroform, and the second solution may be a solution of a drug, such as indomethacin, in methanol.

In a possible implementation form of the first aspect, the core film layer may be provided on top of the lid layer by supplying the slot die head with at least two different materials being fed from at least two different outlets in liquid form, and the slot die head may be heated to a temperature for maintaining the received materials in liquid form. As an example: a hydrophobic polymer, such as PCL, and a solution containing a small molecule drug, such as furosemide in acetone, can be fed come from two different outlets into the slot die heat, which is heated to 80-100 degrees C., where the polymer remains melted and gets blended with the drug solution. This blend is then coated on a substrate while blending continues within the slot die head itself. A cooling or drying process may follow the deposition of the melted film layer.

In a possible implementation form of the first aspect, the shell film layer and the lid film layer are made of the same material, and the shell film layer has a greater thickness than the lid film layer, such as a thickness being at least 1.5 times the thickness of the lid film layer, such as a least 2 times the thickness of the lid film layer, such as a least 5 times the thickness of the lid film layer, such as a least 10 times the thickness of the lid film layer, such as a least 15 times the thickness of the lid film layer. When using the same bio-degradable material for the shell layer and the lid layer, the lid layer will degrade before the shell layer, thereby allowing any active ingredient being part of the core layer to be released from the lid side, while being protected from release through the shell side of the microstructures.

In a possible implementation form of the first aspect, the shell film layer and/or the lid film layer are made of a bio-compatible or bio-degradable or material.

In a possible implementation form of the first aspect, a shell layer may be provided on top of the core layer by supplying a solution of a solvent holding a biodegradable polymer, which is dispensed from the slot die head and deposited as a wet film layer, followed by a heating process for drying the wet film layer. The solution may hold a polymer such as polycaprolactone, PCL, or such polylactic acid, PLA, which may be dissolved in a solvent, such as chloroform or dichloromethane, DCM. The wet film layer may be heated to at least 40 or 50 degrees C. for drying of the wet film layer by evaporation of the solvent.

In a possible implementation form of the first aspect, a shell layer may be provided on top of the core layer by dispensing a biodegradable polymer from the slot die head in melted form and depositing the polymer as a film layer of melted polymer, wherein the slot die head is heated to a temperature for maintaining polymer in melted form. The polymer may be polycaprolactone, PCL, or such polylactic acid, PLA, and the slot die head should be heated to a temperature of at least 80 degrees C. A cooling or drying process may follow the deposition of the melted film layer.

In a possible implementation form of the first aspect, the punching step comprises: applying a pressure to the rigid stamp for a given time period, said pressure having a value being high enough to allow the protrusions of the rigid stamp to penetrate all the way through the plurality of film layers within the given time period. By having the protrusions of the rigid stamp penetrating all the way through the plurality of film layers, the plurality of film layers is divided into a plurality of separated microstructures.

In a possible implementation form of the first aspect, a relatively high pressure is applied to the rigid stamp for said given time period for applying a pressure to the rigid stamp during punching, said pressure having a value of not lower than 6 bar, such as not lower than 7 bar, such as not lower than 8 bar, such as not lower than 9 bar, or such as not lower than 10 bar.

In a possible implementation form of the first aspect, said given time period for applying a relatively high pressure to the rigid stamp during punching is no longer than 3 minutes, such as no longer than 2 minutes, or such as no longer than 1 minute. In a possible implementation form of the first aspect, then for at least a first or start part of said given time period, no external heat is supplied to the film layers. In a possible implementation form of the first aspect, external heat may be supplied to the film layers during a second or end part of said given time period. In a possible implementation form of the first aspect, the external heat may be supplied to the film layers for a relatively short time period being no more than half the given time period, such as no more than 30 seconds. Here, the supplied external heat may be controlled for the film layers to reach a temperature not lower than 40° C.

In a possible implementation form of the first aspect, no external heat is supplied to the film layers during said given time period for applying a relatively high pressure to the rigid stamp during punching. In a possible implementation form of the first aspect, external heat is supplied to the film layers after expiry of said given time period for applying a relatively high pressure to the rigid stamp during punching. This external heat may be supplied for a relatively short time period being no more than half the given time period, such as no more than 30 seconds. The supplied external heat may be controlled for the film layers to reach a temperature not lower than 40° C. In a possible implementation form of the first aspect, then for punching processes for which external heat is supplied to the film layers, the punching is ended by withdrawing of the rigid stamp from the handling substrate when the supply of the external heat has ended.

In a possible implementation form of the first aspect, a relatively low pressure is applied to the rigid stamp for said given time period for applying a pressure to the rigid stamp during punching, said pressure having a value of not higher than 5 bar, such as not higher than 4 bar, such as not higher than 3 bar, or such as not higher than 2 bar. The relatively low pressure may be applied to the rigid stamp for a given time period, which is no shorter than 5 minutes, such as no shorter than 10 minutes, or such as no shorter than 15 minutes. External heat may be supplied to the film layers during at least a part of said given time period for which the relatively low pressure is applied to the rigid stamp, such as during at least half of said given time period, or such as during at least ¾ of said time period. Here, the supplied external heat may be controlled for the film layers to reach a temperature not lower than 50° C., such as not lower than 65° C. Here, the supply of external heat may be stopped before or at the expiry of said given time period for which the relatively low pressure is applied to the rigid stamp, and the applied relatively low pressure to the rigid stamp may be maintained for an extra time period, thereby allowing the heated film layers to cool down to ambient temperatures. In a possible implementation form of the first aspect, external cooling is provided during said extra time period to cool down the heated film layers. In a possible implementation form of the first aspect, for which a relatively low pressure is applied to the rigid stamp for said given time period, the punching is ended by withdrawing of the rigid stamp from the handling substrate when the heated film layers have cooled down for a period.

When punching of several film layers comprising at least a lid layer, a core layer and a shell layer, then punching at a high pressure and at ambient temperatures may draw the shell layer down through the core layer and the lid layer, thereby dividing the film layers into a plurality of separated microstructures, which may be maintained on the deformable surface layer of the handling substrate after withdrawal of the stamp holding the protrusions, or which may be attached within the protrusion when the stamp is withdrawn after the punching. In order to ensure that the lid layer is secured to the end of the drawn shell layer, a short heat fusion may be needed at the end of punching or following the punching. The temperature to be supplied during the heat fusion depends on the materials used of the shell layer and the lid layer. By punching at a high pressure, drawing of the shell layer down through the core layer can be obtained without heating, whereby the overall time needed for the punching and heat fusion is lowered compared to a punching at low pressure, which requires heating of the film layers during a prolonged punching process followed by a period of cooling.

In a possible implementation form of the first aspect, the plurality of microstructures, which are separated during the punching step, may further be separated from the deformable surface layer. In a possible implementation form of the first aspect for which the microstructures hold an outer shell layer, the microstructures may be separated from the deformable surface layer by bonding the outer shell layer of the microstructures onto a release layer. In another possible implementation form of the first aspect for which the microstructures hold an outer shell layer and a lid layer, the microstructures may be separated from the deformable surface layer by being attached within the protrusions of the stamp, and by bonding the lid layer of the microstructures to a release layer. The release layer may be selected from the list consisting of: tape, water soluble polymer layers. The release layer may be water soluble, and the microstructures may be separated from the release layer by putting the release layer in water and then collecting the separated microstructures by filtering.

In a possible implementation form of the first aspect, for which the plurality of microstructures are separated from the deformable surface layer, the handling substrate may hold a deformable surface layer being made of a water-soluble material, and the microstructures may be separated from the deformable surface layer by dissolving the deformable surface layer.

In a possible implementation form of the first aspect, for which the film layer(s) is/are subjected to a punching step performed by use of a rigid stamp having protrusions, the protrusions of the rigid stamp may be made of a material having a hardness being higher than the hardness of the deformable material or deformable surface layer of the handling substrate. The protrusions of the rigid stamp may have a first stiction with regards to the one or more film layer(s) to be punched, the deformable layer may have a second stiction with regards to the one or more film layer(s) to be punched, and the first stiction may be lower than the second stiction. The protrusions of the rigid stamp may be coated with a stiction reducing layer, selected from the list consisting of: fluoropolymers, such as polytetrafluoroethylene (PTFE), fluorosilanes, such as per-fluoro-decyl-trichlorosilane (FDTS). The protrusions that allows for the generation of the plurality of microstructures may be formed so that each microstructure has an outer shape comprising a width and a height of ≤9000 μm, such as ≤5000 μm, such as ≤2500 μm, such as ≤1000 μm, such as 900 μm, such as ≤800 μm, such as ≤700 μm, such as ≤600 μm, such as ≤500 μm, such as ≤400 μm, such as ≤300 μm, such as ≤250 μm, such as ≤200 μm, such as ≤150 μm, such as ≤100 μm, such as ≤50 μm.

The rigid stamp with protrusions may be made of a metal or metal alloy, such as nickel, aluminum, stainless steel, iron or brass, or the made of silicon or glass. In an embodiment the stamp with protrusion is made of nickel, Ni, and may be produced by a process of electroplating nickel on a silicon template followed by removal of the silicon template. Such a process is described in examples 3 and 7 of US patent application No. 2016/0206513.

In a possible implementation form of the first aspect, the thickness of each of the provided film layers is in the range of 5 to 200 μm.

In a possible implementation form of the first aspect, the handling substrate holds a deformable surface layer having a thickness in the range of 5 to 200 μm.

In a possible implementation form of the first aspect, the deformable surface or deformable surface layer of the handling substrate is subjected to an oxygen plasma treatment prior to depositing the one or more film layer(s).

In a possible implementation form of the first aspect, the handling substrate or the deformable surface layer of the handling substrate is made of a thermosetting material, such as Polydimethylsiloxane PDMS.

In a possible implementation form of the first aspect, the handling substrate is substantially flat and made of a flexible material suitable for rolling.

In a possible implementation form of the first aspect, the handling substrate is made of a material selected from the list: mylar, paper, plastic, metal, foil or combinations thereof.

In a possible implementation form of the first aspect, the handling substrate is a deformable Polyethylene sheet. The thickness of the Polyethylene sheet may be about or no less than 100 μm.

In a possible implementation form of the first aspect, the handling substrate or the deformable surface layer of the handling substrate is made of a silicon material, or a rubber material, or Aluminum sheet.

In a possible implementation form of the first aspect, the handling substrate has a width in the range of 5 to 50 cm, such as in the range of 15 to 16 cm.

In a possible implementation form of the first aspect, wherein after the one or more film layers have been provided by slot die coating and before performing any punching, the handling substrate holding one or more film layers are rolled for storage.

In a possible implementation form of the first aspect, wherein the handling substrate is made of a deformable material, the punching process may be followed by rolling the handling substrate holding the punched microstructures before any release of the microstructures from the handling substrate.

According to a second aspect there is provided a microstructure for delivery of an active ingredient to a user, said microstructure being produced in accordance with one or more implementations of the first aspect, which implementations holds a punching step for generation of a plurality of microstructures, which microstructures have a core film layer holding the active ingredient.

The foregoing and other objects are achieved by the features of the independent claims. Further implementation forms are apparent from the dependent claims, the description and the figures. These and other aspects of the present disclosure will be apparent from the embodiments described below.

### BRIEF DESCRIPTION OF THE DRAWINGS

In the following detailed portion of the present disclosure, the aspects will be explained in more detail with reference to the example embodiments shown in the drawings, in which:

FIG. 1 is a schematic block diagram illustrating the basic structure of a system for manufacturing a plurality of microstructures according to an example embodiment;

FIG. 2 shows is a block diagram illustrating the functional components of a slot die coating system for coating a film layer on a substrate according to an example embodiment;

FIGS. 3a and 3b illustrate the design of a cylindrically protrusion for a stamp according to an example embodiment;

FIG. 3c shows a SEM micrograph of a single protrusion of a nickel stamp, which protrusion is cylindrically shaped for forming a cylindrically shaped microstructure according to an example embodiment;

FIG. 3 d shows SEM micrographs of nickel stamps having different shaped protrusion;

FIGS. 4a-4d are schematic illustrations of the steps of punching and harvesting microstructures from a 2-layered film according to an example embodiment;

FIGS. 5a-5c are schematic illustrations of the steps of punching and harvesting microstructures from a 3-layered film according to an example embodiment;

FIGS. 6a-6d are schematic illustrations of different manufacturing processes of microstructures according to example embodiments;

FIGS. 7a-7d are flow diagrams corresponding to the manufacturing processes of FIGS. 6a-6d according to example embodiments;

FIG. 8 is a schematic illustration of three 3-layered microstructures with different thicknesses of a middle layer holding an active ingredient according to example embodiments;

FIG. 9 is a schematic illustration of punching and harvesting of microstructures stored on a handling substrate according to an example embodiment;

FIG. 10 is a schematic illustration of punching and harvesting of microstructures stored in protrusions of a punching stamp according to an example embodiment;

FIG. 11 is a schematic illustration of microstructures with different number of film layers holding an active ingredient according to example embodiments;

FIG. 12 is a schematic illustration of different microstructures provided with a mucoadhesive material according to example embodiments;

FIG. 13 is a schematic illustration of a microstructure manufactured for a targeted release of an active ingredient according to an example embodiment;

FIG. 14 is a schematic illustration of a microstructure manufactured for a sustained release of an active ingredient according to an example embodiment;

FIG. 15 is a schematic illustration of a microstructure manufactured for a controlled release of an active ingredient according to an example embodiment;

FIG. 16 is a schematic illustration of a microstructure manufactured for a pulsatile release of an active ingredient according to an example embodiment;

FIG. 17 is a schematic illustration of a microstructure manufactured for a burst release of an active ingredient according to an example embodiment;

FIG. 18 is a schematic illustration of harvesting of microstructures from a water-soluble layer according to an example embodiment;

FIGS. 19a and 19b are schematic illustrations of surface treatment of microstructures according to example embodiments; and

FIGS. 20a and 20b are schematic illustrations of bonding or fusion of two microstructures according to example embodiments.

### DETAILED DESCRIPTION

### General Overview of the Process

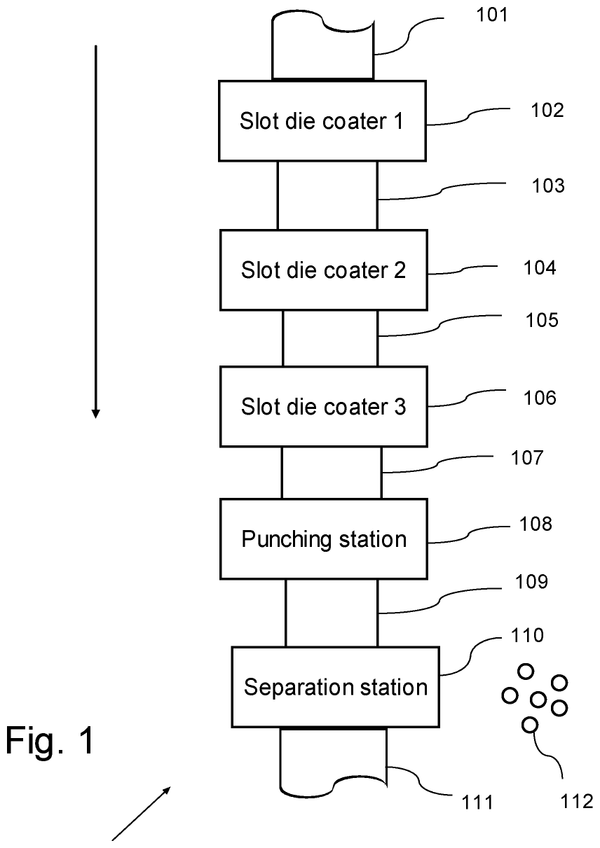

The present disclosure relates to a versatile, high-throughput technique of encapsulating different cargos in an anisotropic particle in for example two steps. The technique is based on the combination of two scalable processes. These techniques are slot die coating and punching of microstructures. Slot die coating can produces films of precise thickness and uniformity in a continuous fashion. As many film layers as desired can be stacked upon each other by subsequent depositions. After the production of film layers, which may have different functionalities, and which may hold at least one layer containing an active ingredient, punching for forming microstructures is performed. This punching may allow the fabrication of non-spherical micro/nanoparticles with anisotropic and/or core-shell properties. A schematic of the overall process is shown in FIG. 1.

Diverging from the viewpoint of considering microstructure particle material as a liquid/semi-liquid ink, which is patterned such as in the conventional lithographic and printing techniques, the microstructure particle material can be viewed as a plastic film that can be deformed and cut to create microstructure particles with desired structures and features. In traditionally available methods, the particle shaping occurs by liquid flowing onto or into a mold. In the proposed technique, the materials for each layer are deposited as a film layer by slot die coating, where the deposited film layer may be a wet film layer, which may be partially or fully dried. Subsequently a mold or stamp of a rigid material, which may be heated, may be used in a punching process, in which the stamp cuts through the stack of film layers, while shaping the stacked layers into individual microstructures or particles.

Slot Die Coating

Slot die coating is a versatile process that is widely used for the production of thin and uniform films ranging from tens of nanometers to hundreds of microns in thickness. Some of the benefits of slot die coating include deterministic thickness control (often described as being “pre-metered”), high precision, continuous operation, absence of contact with the sample, and high scalability to coating speeds of the order of up to 400-700 m/min and areas ranging from a few cm² up to many m² (depending on the material/process). Slot die coating machines may achieve their coatings by delivering the desired coating material onto the substrate through a highly precise deposition vessel known as the slot die head.

The slot die head may serve to precisely and uniformly distribute the coating fluid along the entirety of the desired coating width. This is achieved by pumping ink from a sample reservoir into the slot die head, where it may be redistributed along the length of a long internal manifold. The ink may subsequently be guided to the coating edge of the slot die head via a thin metal cutout known as a shim. The shape of the shim can be altered to adjust the coating width and stripe pattern. This makes slot die coating a tremendously scalable, controllable, high throughput technique with applications ranging from lab to pilot to industrial scale.

The deposition of the coating fluid onto the substrate from the slot die head may be governed by a wide array of process parameters, as well as the geometry of the slot die head and its orientation with respect to a substrate being coated. The wet film thickness may directly be controlled by the ink pump rate, substrate speed, sometimes also referred to as “coating speed”, and total coating width of a given process. However, it should be noted that the final film thickness after drying may be significantly less due to removal of a solvent, which may be required.

While the coating thickness may be easily controlled, the process must be further optimized to deliver a high-quality film at the desired thickness. Unoptimized processes are liable to produce poor quality films with a variety of possible defects, e.g. runny stripes, ribbed stripes, pinholes in the coated layer, etc. As such, optimizing a slot die coating process generally involves identifying a combination of parameters that provides a film of the desired thickness without succumbing to the formation of film defects. The matrix of combinations that satisfies these criteria is highly material- and process-depending and is typically referred to as the “stable coating window” of the process.

Additional process parameters can be manipulated to influence the coating quality. These may include the height and angle of the slot die head with respect to the substrate, the viscosity and affinity of the ink towards the desired substrate material, the surface energy of the substrate to improve its wettability, and the heating of the substrate to influence drying, among others.

Slot die coating can be used to precisely fabricate films having different functionalities with at least one layer containing the active ingredient. As an example, for active ingredient delivery applications, different layers such as: a) an outermost biodegradable layer that forms a protective shell; b) an active ingredient layer containing an active ingredient slurry or matrix; and c) a lid layer that may be removed by active or passive triggers, can be deposited using slot die coating.

For microstructures or particles having a film layer holding an active ingredient, such as a drug, the precision of the active ingredient layer may control the accuracy in active ingredient dosage in patients. For microstructures or particles having a lid layer covering the active ingredient layer, the precision of the lid layer may control the timing of active ingredient delivery. The combination of a precise protective shell layer and a lid layer allows for control over the place and profile of active ingredient release in the body of a patient. Thus, for a special case of oral active ingredient delivery, the shell layer may protect the active ingredient in the harsh acidic environment of the stomach. Once the microstructure or particle arrives in the intestine, the possible embedding of the active ingredient in adhesive polymer may help in better mucoadhesion to the intestinal walls and the pH-sensitive lid layer may dissolve in the basic environment in the intestine leading to a targeted delivery of the active ingredient.

Punching of Microstructures or Particles

As described previously, a novel perspective in punching of microstructures or particles is the manipulation of the material layer (ML) as a deformable film. This perspective takes inspiration from the high throughput manufacturing technique called punching or blanking, that has been used for metal sheet forming for hundreds of years. Blanking and punching are shearing processes in which a punch and die are used to modify webs. Blanking is the process where the cut part is used as the main device part, while punching is the process where the leftover sheet without the cut parts is the main device part. Here we make no distinguish between the two processes and even though we are interested in the cut part, we call the process “punching” nonetheless. The conventional punching/blanking process includes three basic steps: 1. A sheet metal is placed on a rigid die with a convex structure or opening. 2. The punch and the metal sheet are brought into physical contact by a high force. The insertion of punch into the die leads to deformations and stresses on the sheet metal. The maximum stress on the sheet metal happens right underneath the edges of the punch. During this step, when the maximum stress exceeds the break strength of the metal, part of the metal plate right underneath the convex mold structure is cut off from the neighboring metal by the punch. 3. Finally, the punch and the die are separated. The two biggest differences between the conventional punching/blanking technique and the process of punching of microstructures or “micropunching”, are the utilization of a deformable die and a hollow punch. Punching of microstructures or “micropunching” may be described as a process of producing particles from a device layer lying on a deformable layer. This stack of the deformable layer and the device layer may be punched by a rigid punch/mold/stamp holding protrusions defining cavities. Two processes may occur simultaneously during “micropunching”: the cutting or indentation of the device layer by the hollow protrusions of the mold/stamp and the final punching of the device layer, which may lead to the fabrication of microstructures or particles of controlled shape, size and features. The deformation of the device layer may in most cases be by shear, facilitated by the deformable layer and sometimes also by temperature. When the deformable layer is substituted with a hard layer under device layer(s), e.g., Si wafer, glass slides etc., as in conventional imprint lithography, the device layer cannot be punched through. Thus, a residual layer remains connecting the patterned film instead of fabrication of discrete particles. This problem is solved in “micropunching” with the help of a deformable layer, where the stamp can get through the device layer, finally punching the microstructures or particles from the rest of the device layer.

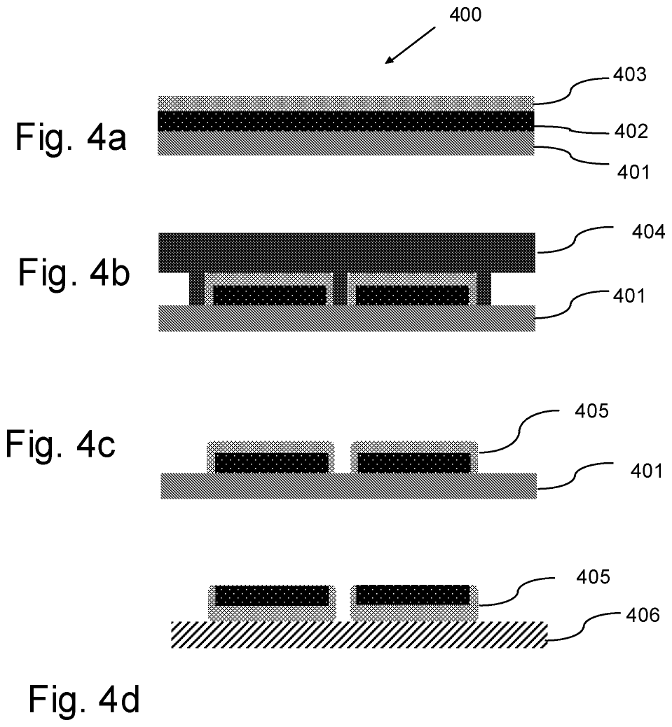

There is a third process, which may occur when the device layer material is ductile and yields before it is broken completely by a punching process. This step may be called the “microdrawing” version of metal sheet manufacturing technique called “deep drawing” or just “drawing”. Deep drawing is a metal sheet forming process in which a sheet metal blank is radially drawn or stretched into a forming die by the mechanical action of a punch. FIG. 4b demonstrates “microdrawing” of the device layer material around the walls of a micropatterned rigid mold or stamp, such as a Ni stamp. When combined, “micropunching” and “microdrawing” can create shell like isolated structures on a single layer. Under the application of pressure during the micropunching process, the deep drawing or “microdrawing” process occurs around the micropatterned walls of the rigid stamp or mold. When the device layer comprises several film layers, once the pressure applied on the walls of the stamp extends the ultimate tensile strength of the device layer, an outermost film layer of the device layer get punched around one or more inner film layers to form walls around the inner film layers, thereby forming of microstructures or microparticles. The drawing and the punching processes in “micropunching” and “microdrawing” can be facilitated by the application of heat.

Stamp or Mold Preparation for Micropunching

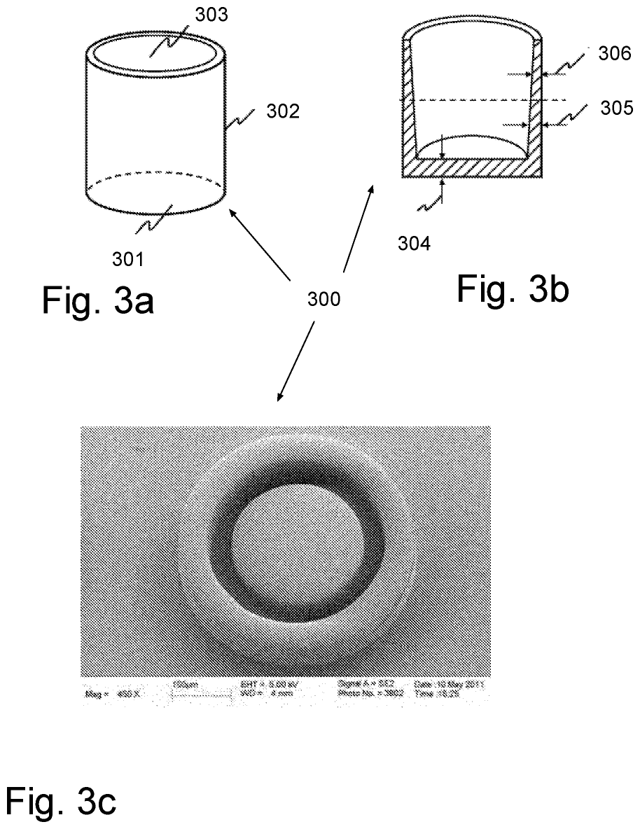

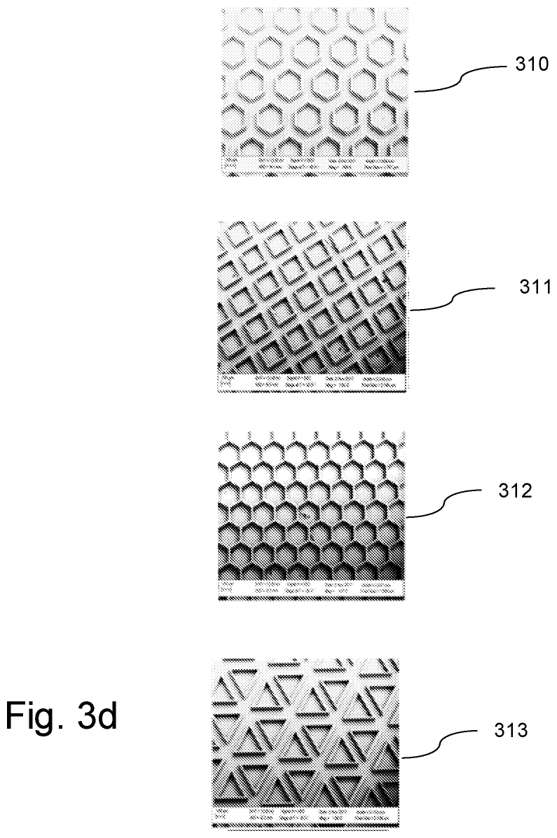

“Micropunching” or punching for generation of microstructures or microparticles requires a rigid and robust mold or stamp as a punch tool. The stamp or mold may include protruding walls around a cavity of desired size and shape to cut and shape the device layer, which may include a number of polymer film layers. The mold or stamp can be made of different materials like Ni, tungsten, steel or other metals or their alloys, thermoset polymers like crosslinked photoresists and other hard polymers like graphene or metal containing polymers.etc. The mold or stamp should be rigid enough to tolerate the pressure and temperature requirements of the “micropunching” process. The walls of the mold or stamp should be thin enough to result in punching but not too thin that they act as a blade, thereby limiting the drawing process required for forming a shell. The mold or stamp may have anti-stiction coating or may be made of a material that has anti-stiction or superhydrophobic properties. The mold or stamp should preferably not have rough sidewalls and negative tapering. This permits easy harvesting of the microstructures or microparticles after punching and demolding. The mold or stamp should have the desired shape and height in nanometer and micrometer size ranges. For example, a Ni punching mold or stamp may be fabricated by using DEEMO (Dry etching, electroplating and molding) process. The Ni mold or stamp may have protruding walls around a cavity of desired size and shape, as shown in FIG. 3. A molecular layer of Perfluorodecyltrichlorosilane (FDTS) may be deposited on the Ni mold using molecular vapor deposition (MVD) in some cases. This may improve the separation of the mold or stamp from the molded device layer, which may be polymer fil layers, by reducing the adhesion forces between the two parts.

Example of fabrication of rigid stamp for punching of microstructures or microparticles For the punching of microstructures, a stamp having protrusions with vertical or near vertical sidewalls may be preferable, see FIG. 3b. Negative slopes are typically avoided because of the risk of trapping the polymer in the stamp, and also because it hinders the removal of the stamp after completed processing. For the punching of the microstructures a fabrication process for nickel stamps having protrusions with positive sidewall slopes is used. This should support the enclosure of the core layer by the barrier layer during the punching process.

The stamp fabrication is based on electroplating of nickel on a silicon template followed by removal of the template. First, 500 nm of wet silicon oxide are deposited on a standard 4-inch SC silicon wafer during 50 min in a LPCVD furnace (Tempress, MD Vaassen, the Netherlands) at 1100° C.

Next, a first step of photolithography is performed to allow patterning of the silicon oxide. For this purpose, the wafer is coated with hexamethyldisiloxane (HMDS) and a 1.5 μm thick film of positive photoresist AZ5214e (Clariant GmbH, Wiesbaden, Germany) is applied by spin coating on a Maximus 804 spin coating equipment (ATMsse GmbH, Singen, Germany). The photoresist is soft-baked for 90 s at 90° C. on a hotplate and exposed through a photolithographic mask (Delta Mask B. V., G J Enschede, the Netherlands) in hard contact mode with a dose of 35 mJ/cm² in a MA6/BA6 UV mask aligner (Karl-Suss, Garching, Germany) equipped with an i-line filter (365 nm, 20 nm FWHM).

The exposed photoresist was developed for 60 s in AZ351 developer (Clariant) in a (1:5) dilution with water. The photoresist serves as etch mask for the patterning of the underlying oxide layer. The etching of the silicon oxide is performed in BHF for 10 min followed by stripping of the photoresist mask in Acetone. A second step of photolithography identical to the one described above is performed consisting of HMDS, spin coating, UV exposure with a different photolithographic mask and development.

Next, two steps of deep reactive ion etching (DRIE) of the silicon bulk material are performed in a Pegasus DRIE system (STS, Newport, UK). A BOSCH process at 0° C. is used, switching between a passivation cycle with a gas flow of 150 sccm C4F8 (pressure mTorr, coil power 2000 W, platen power 0 W, cycle time 2 s) and an etching cycle with gas flows of 275 sccm SF₆ and 5 sccm O₂ (26 mTorr, 2500 W, 35 W, 2.4 s). In the first etching step to a depth of 20 μm, the photoresist layer serves as etch mask to obtain the pattern corresponding to the outer circumference of the protrusions. After this step, the photoresist is removed in stripped in acetone followed by cleaning in oxygen plasma. In the second etching step to a depth of 80 μm, the patterned silicon oxide serves as etch mask to obtain the pattern corresponding to the reservoir of the protrusions. After the DRIE, the silicon oxide etch mask is removed in BHF. The seed layer for the electroplating process consisting of 20 nm Ti and 300 nm Au is deposited in a CMS-18 sputter system (Kurt. J. Lesker Company, Jefferson Hills, USA). Next, 500 μm of Ni are electroplated on the metal coated template on a microform.200 Nickel electroplating machine (Technotrans, Sweden) with a plating bath of aqueous nickel sulphamate, boric acid and sulfamic acid at 51° C. and pH 3.5-3.8. The current is linearly increased to 0.5 A during 15 min followed by ramping to 1.5 A during additional 15 min. The current is maintained at 1.5 A for 30 min and increased to the final value of 6.5 A during 15 min. There, the electroplating is continued for approximately 3 h until a final setpoint charge of 26.8 Ah is reached. The electroplating step is followed by the removal of the silicon template in 28 wt. % KOH at 80° C. during approximately 10 h resulting in a Ni stamp coated with Au.

A stamp with 4×4 array of 20×20 patches of microprotrusions is designed. FIG. 3c shows SEM micrograph of a Ni stamp feature for the definition of one protrusion. In total, there are 6400 protrusions per stamp. An individual protrusion consists of two parts, an inner disc and an outer ring structure. The total width of the protrusions is 300 μm. The wall and the outer ring thicknesses are 40 μm and 30 μm, respectively. The stamp is fabricated as described above and then coated with teflon. The height of the outer ring is 80 μm and the one of the inner disc is 65 μm.

### DESCRIPTION OF EMBODIMENTS

FIG. 1 is a schematic block diagram illustrating the basic structure of a system 100 for manufacturing a plurality of separate microstructures 112 according to an example embodiment. A handling substrate 101, to which to which a number of film layers are to be coated, is fed into a first slot die coating station 102, where a first film layer 103 is coated on the substrate 101. The substrate 101 with film layer 103 is then fed to a second slot die coating station 104, where a second film layer 105 is coated on top of the first film layer 103. The substrate 101 with film layers 103 and 105 is then fed to a third slot die coating station 106, here a third film layer 107 is coated on top of the second film layer 105. The substrate 101 with film layers 103, 105 and 107 is then fed to a punching station 108, where the film layers are punched into microstructures 109, which may be stored on the handling substrate 101. The handling substrate 101 carrying the punched microstructures may then be fed to a separation 110, in order for the microstructures to be separated from the substrate 101 into separate microstructures or micro-particles 112. In FIG. 1, reference numeral 111 refers to the remaining substrate after punching and separation of microstructures.

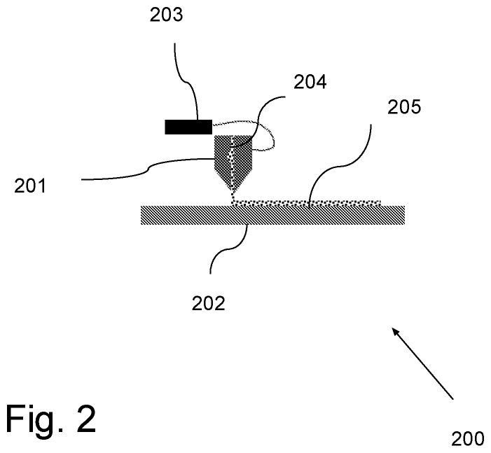

FIG. 2 shows is a block diagram illustrating the functional components of a slot die coating system 200 for coating a film layer 205 on a handling substrate 202 according to an example embodiment. The slot die coating has a slot die head 201, which is supplied with the material to be coated 204 from a coating liquid pump 203. The slot die system 200 may hold a sheet coater or a roll-to-roll system, not shown in FIG. 2, for supporting the handling substrate 202. The company FOM Technologies delivers sheet-based coater systems, such as the FOM vectorSC system and the FOM alphaSC system, and roll-to-roll based systems. such as the FOM moduloR2C.

FIGS. 3a and 3b illustrate the design of a cylindrically protrusion 300 for a stamp according to an example embodiment. The protrusion has a bottom 301, one or more sidewalls 302, and an opening 303. The bottom has a thickness 304, and the sidewalls 302 have different thicknesses 305 and 306, such that the interior of the protrusion 300 has a tapered shape with positive sidewall slopes and with the largest inner diameter at the opening 303. This tapered shape may support the enclosure of the core film layer by the shell film layer during the punching process.

FIG. 3c shows a SEM micrograph of a single protrusion 300 of a nickel stamp, which protrusion is cylindrically shaped for forming a cylindrically shaped microstructure according to an example embodiment. In total, there are 6400 protrusions 300 per stamp. An individual protrusion 300 consists of two parts, an inner disc and an outer ring structure, with the inner disc formed by in the interior of the sidewalls 302 and the outer ring structure formed by the exterior of the sidewalls 302. The total width of the protrusion 300 is 300 μm. The sidewalls 302 have a thickness of 40 μm at the bottom and a thickness of 30 μm, at the opening 303. The stamp is fabricated as described above and then coated with teflon. The height of the outer ring or outer sidewalls is 80 μm and the one of the inner disc or inner sidewalls is 65 μm.

FIG. 3 d shows SEM micrographs of nickel stamps having different shaped protrusion. The first stamp has separated hexagonal shaped protrusions 310, the second stamp has separated square shaped protrusions 311, the third stamp holds a mesh of hexagonal shaped protrusions 312, and the fourth stamp has separated triangular shaped protrusions 313.

FIGS. 4a-4d are schematic illustrations of the steps of punching and harvesting microstructures from a 2-layered film 400 according to an example embodiment. FIG. 4a shows a handling substrate 401, which is made of deformable material, coated with a core film layer 402 being the first layer, which may hold an active ingredient, with the core film layer coated with a shell layer 403, which is the second layer. FIG. 4 b shows punching of the film layers 402, 403 by use of a rigid stamp 404 having protrusions with sidewalls. The protrusions of the stamp 404 penetrates fully through the two film layers 402, 403 to reach the deformable substrate 401. The depth of the sidewalls of the protrusions of the stamp is a bit larger than the combined thickness of the two film layers 402, 403, whereby the protrusions of the stamp 404 will press against and into the deformable substrate, to thereby assure total separation of the film layers 402, 403 into separated microstructures 405, see FIG. 4c. In FIG. 4c, the stamp 404 is removed leaving the microstructures 405 on the handling substrate 401. FIG. 4d shows an embodiment for removing the microstructures 405 from the substrate 401. The top side of the microstructures 405, which is now formed by the shell layer 403 can be bonded or secured to a release layer 406, which for example may be a tape or a water-soluble polymer layer. When using a water-soluble layer, the microstructures 405 can be released by cutting a piece of the polymer layer and put into a glass of water.

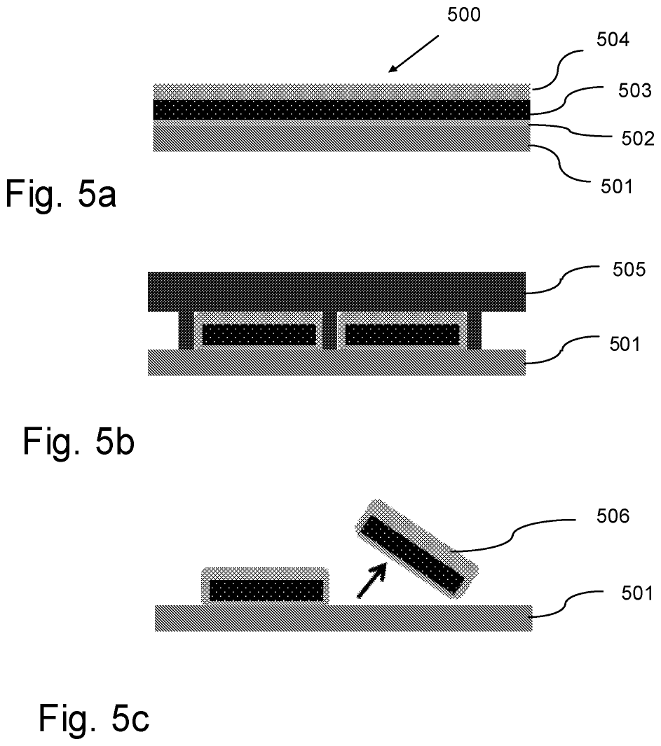

FIGS. 5a-5c are schematic illustrations of the steps of punching and harvesting microstructures from a 3-layered film 500 according to an example embodiment. FIG. 5a shows a handling substrate 501, which is made of deformable material, coated with a lid film layer 502 being the first layer, which is coated with a core film layer 503 being the second layer, which may hold an active ingredient, with the core film layer coated with a shell layer 504, which is the third layer. FIG. 5 b shows punching of the film layers 502, 503 and 504 by use of a rigid stamp 505 having protrusions with sidewalls. The protrusions of the stamp 505 penetrates fully through the three film layers 502, 503 and 504 to reach the deformable substrate 501. Also there, the depth of the sidewalls of the protrusions of the stamp is a bit larger than the combined thickness of the three film layers 502, 503 and 504 whereby the protrusions of the stamp 505 will press against and into the deformable substrate, to thereby assure total separation of the film layers 502, 503 and 504 into separated microstructures 506, see FIG. 5c. In FIG. 5c, the stamp 505 is removed leaving the microstructures 506 on the handling substrate 501. The core layer of the microstructures 506 is fully covered by the shell layer on top and sides and by the lid layer at the bottom. The microstructures 506 can be stored on the handling substrate 501 to be removed when to be used.

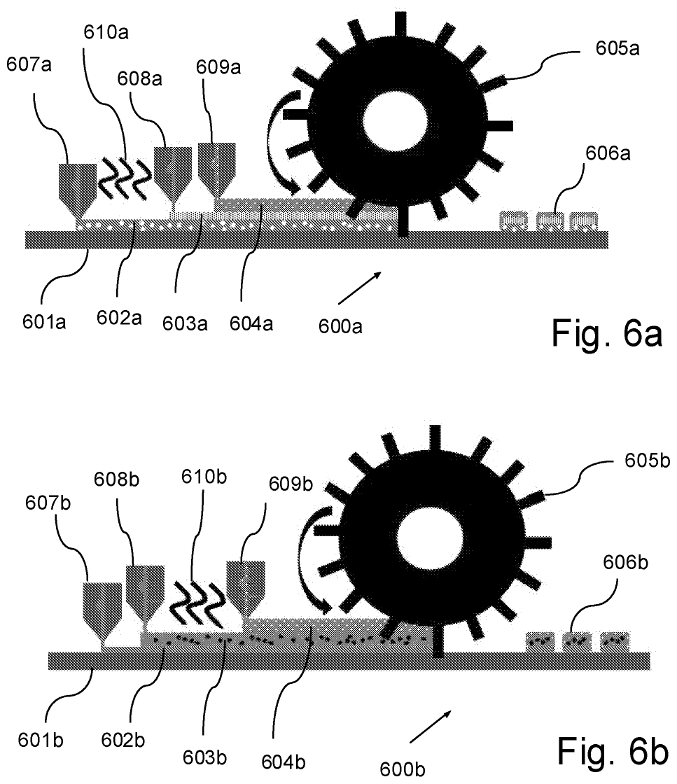

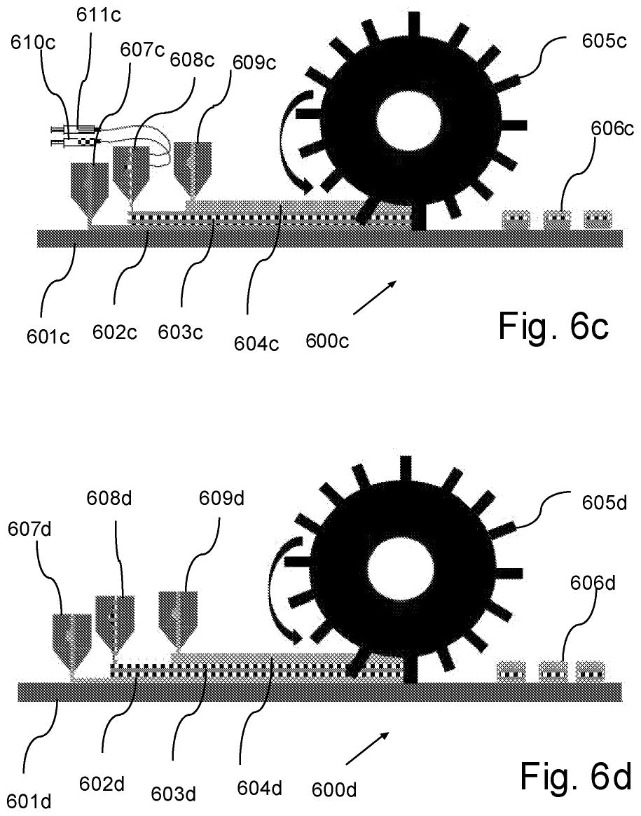

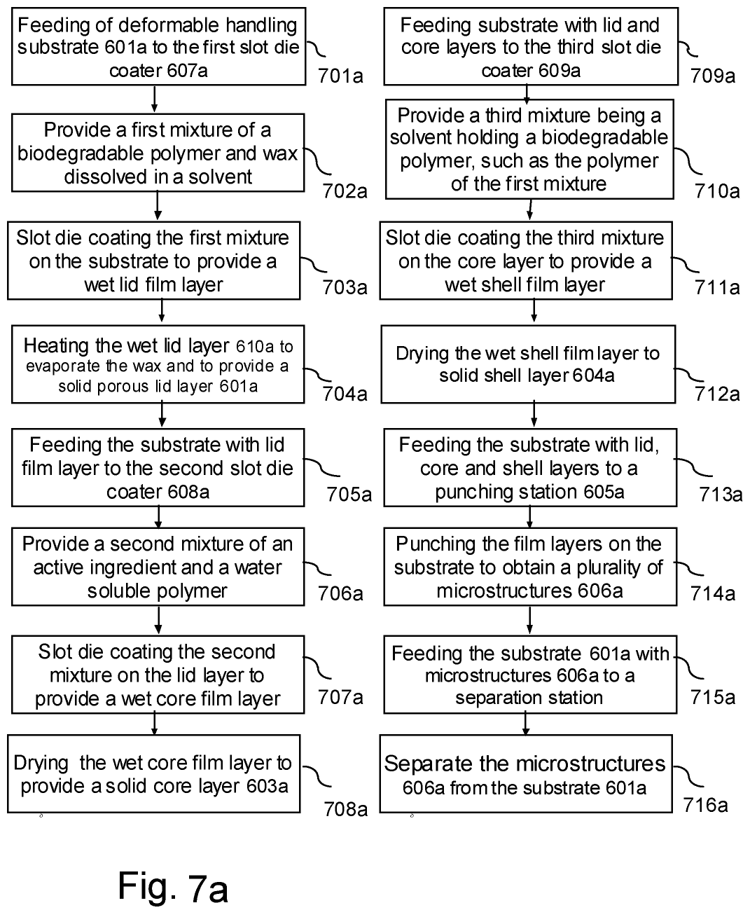

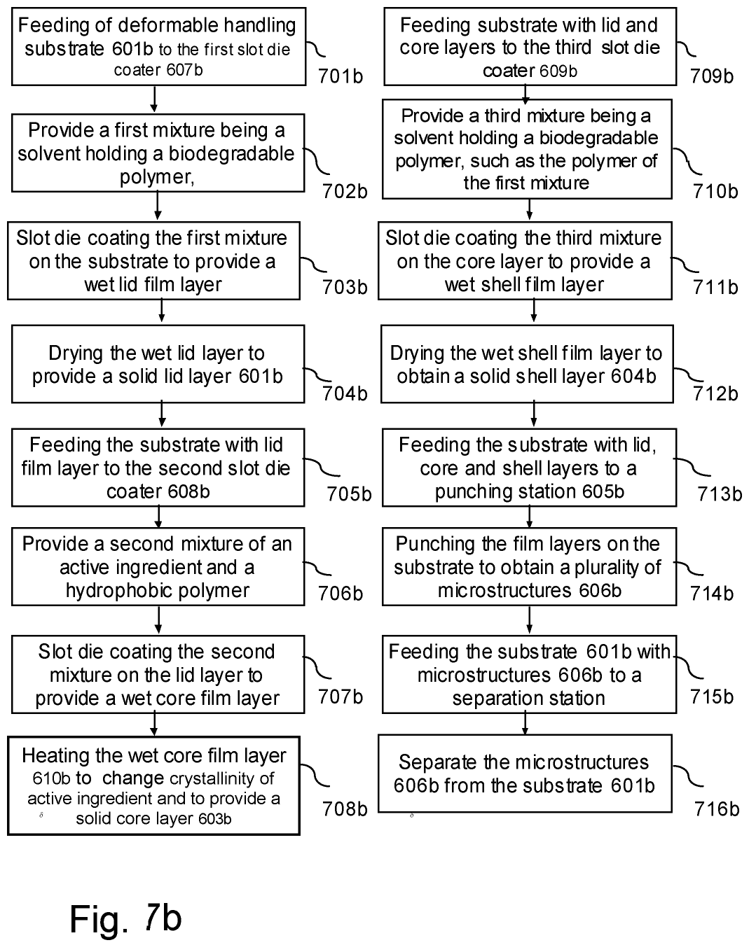

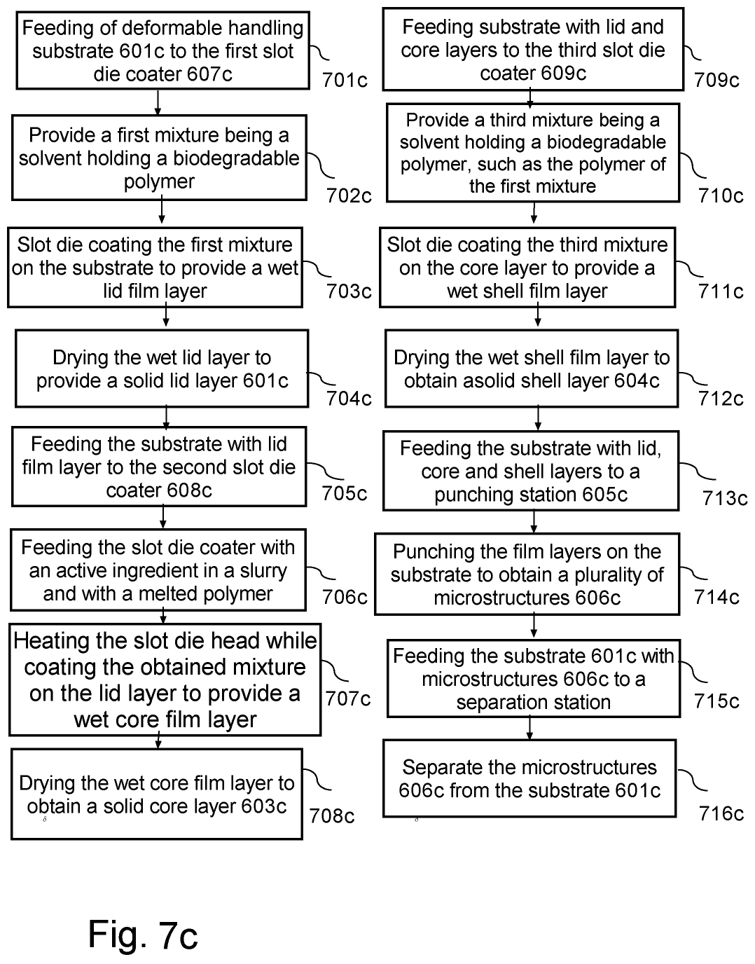

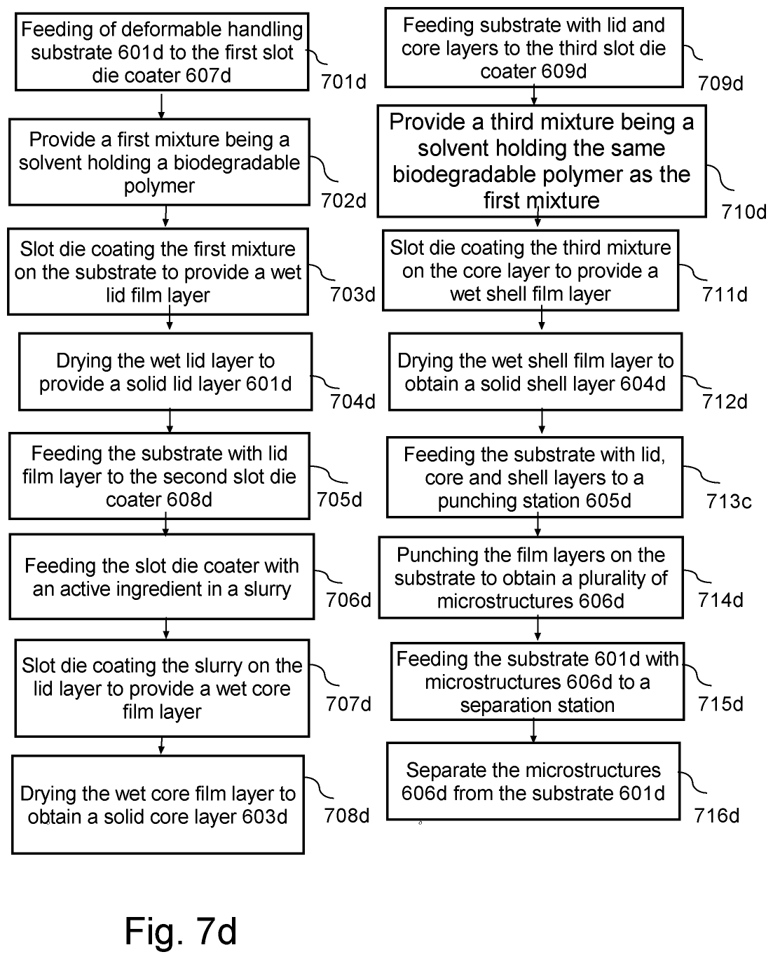

FIGS. 6a-6d are schematic illustrations of examples of different manufacturing processes of microstructures according to example embodiments, and FIGS. 7a-7d are flow diagrams corresponding to the manufacturing processes of FIGS. 6a-6d.

FIGS. 6a and 7a illustrate a first example 600a. Here, the handling substrate 601a is made of a deformable material, which is a polyethylene sheet having thickness in the range of 100 μm to a few mms. The substrate 601a is fed to a first slot die coater 607a, step 701a.

For the first layer a first mixture is provided, step 702a, which contains a biodegradable polymer like polycaprolactone, PCL, or polylactic acid, PLA, and another solid that can sublime or has lower boiling point than PCL or PLA. This solid can be camphor wax. The wax and PCL or PLA polymer are dissolved in a solvent, such as dichloromethane, DCM, chloroform, acetone, and methanol. The first mixture is fed to the first slot die coater 607a to be slot die coated onto the deformable substrate 601a to provide a coated wet film layer of a thickness in the range of 2-50 μm, 602a and step 703a. The deposited material is heated at 40-50 degrees and the wax is sublimed instantaneously or evaporated to give a porous lid film layer, 610a and step 704a. The dried polymer lid film layer is oxygen plasma treated for 30 sec or in other ways to make the lid layer hydrophilic for the next layer, being a core film layer holding an active ingredient.

The substrate 601a with the lid film layer 602a is now fed to a second slot die coater 608a, step 705a. For the second layer a second mixture is provided, step 706a, which contains an active ingredient being a drug such as furosemide and a water-soluble polymer, such as poly acrylic acid, PAA, since furosemide has bad solubility in water. This mixture is prepared by mixing two solutions and making a blend: PAA in water and then furosemide solution in acetone, step 706a. The second mixture is fed to the second slot die coater 608a to be slot die coated onto the first layer into a thickness of 50-100 μm, 603a and step 707a. The core film layer with the active ingredient is then allowed to dry, which may be done in a short time by using infrared, IR, heating or under a hot furnace at 40-50° C. within one minute or more, step 708a. In another embodiment the active ingredient is temperature sensitive like protein etc. Here, the second mixture may be provided by physically mixing the protein with a powdered polymer with low melting point like bee wax, etc. This second mixture is sent through a heated syringe to the slot die head 608a and slot die coated on a cold counter-roller/plate where the melted mixture solidifies and make a coating of a core film layer holding the active ingredient. In yet another embodiment, the core layer with the active ingredient can also be coated without any polymer matrix by making the active ingredient slurry, coating it and letting the film dry, which may take up to for 1-5 hours, or which may be done in a short time by using infrared, IR, heating or under a hot furnace at 40-50° C. within one minute or more. This will allow maximum loading of active ingredient in the punched microparticles.

The substrate 601a with the lid film layer 602a and the core film layer 603a is now fed to a third slot die coater 609a, step 709a. For the third layer a third mixture is provided, step 710a, which contains a biodegradable polymer like polycaprolactone, PCL, or polylactic acid, PLA, dissolved in a solvent, such as dichloromethane, DCM, chloroform, acetone, and methanol and then coated as liquid. The third mixture is fed to the third slot die coater 609a to be slot die coated onto the second layer to provide a wet shell film layer of a thickness of 10-100 μm, 604a and step 711a. The shell film layer is then allowed to dry, which may be done in a short time by using infrared, IR, heating or under a hot furnace at 40-50° C. within one minute or more, step 712a. In another embodiment, then if the active ingredient is not so temperature sensitive like the protein case described above, and the active ingredient is robust at the melting temperature of the shell layer polymer, which is the case when using furosemide as the active ingredient, then the biodegradable polymer may be supplied in melted form to the third slot die coater 609a, to be coated onto the second core layer. The melted polymer is dried instantaneously, when the polymer is deposited. It is noted that the deposited shell layer 604a is thicker than the lid layer 602a. As an example, the lid layer may be about 10 μm and the shell layer about 50 μm, while the core layer may be about 75 μm.

After the deposition of all the film layers, the deformable substrate 601a holding the lid film layer 602a, the active ingredient core film layer 603a and the shell layer 604a is fed to a punching station, 605a and step 713a. At the punching station 605a the film layers 602a, 603a, 604a are punched by a Ni Stamp having protrusions with a depth of 100-200 μm and a diameter of 50-500 μm either by plate to plate or roll to roll or roll to plate forms to obtain separate microstructures 605a on the substrate 601a, step 714a. For the above example of thicknesses of the film layer, the depth of the protrusions should be larger than 135 μm, such as about 150 μm.

The punching may be performed as a plate to plate punching at about 4 bars pressure for 5 minutes with heat supplied to ensure drawing of the shell layer around the core layer, and for shell layers based on PCL, heat is supplied during the punching to ensure the temperature can be kept at 60-70° C., while for PLA based shell layers, heat is supplied during the punching to ensure the temperature can be kept at 90-120° C. In another embodiment, the punching is performed as a roll to roll punching at a high pressure such as about or above 10 bars within a short time of about 1 minute and without heating during the period of high pressure. The high-pressure period may be followed by a short period of about 30 seconds for flash heating the lid layer to the shell layer, which has been punched and drawn around the core layer down to the lid layer. For PCL based shell layers, the flash heating should be about 60-70° C. and for PLA based shell layers, the flash heating should be about 90-120° C.

Finally, the substrate 601a holding the punched microstructures or microparticles 606a, may be fed to a separation station, step 715a, to be separated from the substrate 601a, step 716a. In some embodiments, then after the punching process the microstructures may be stuck into the protrusions of the punching stamp, and the separation of the microstructures 606a will include a separation of the microstructures from the protrusions of the stamp.

FIGS. 6b and 7b illustrate a second example 600b. Here, the handling substrate 601b is made of a deformable material, which is a polyethylene sheet having thickness in the range of 100 μm to a few mms. The substrate 601b is fed to a first slot die coater 607b, step 701b.

For the first layer 602b a first mixture is provided, step 702b, which contains a biodegradable polymer like polycaprolactone, PCL, or polylactic acid, PLA, a pH sensitive polymer such as Eudragit or a mucoadhesive such as chitosan, which may be dissolved in a solvent, such as dichloromethane, DCM, chloroform, acetone, and methanol and then coated as liquid. The first mixture is fed to the first slot die coater 607b to be slot die coated onto the substrate 601b to provide a wet lid film layer of a thickness of 2-50 μm, 602b and step 703b. The lid film layer is then allowed to dry, which may be done in a short time by using infrared, IR, heating or under a hot furnace at 40-50° C. within one minute or more, step 704b. In another embodiment, then the biodegradable polymer may be supplied in melted form to the first slot die coater 607b, to be coated onto the substrate 601b. The melted polymer is dried instantaneously, when the polymer is deposited. This first film layer 602b acts as the lid film layer.

The substrate 601b with the lid film layer 602b is now fed to a second slot die coater 608b, step 705b. For the second layer a second mixture is provided, step 706b, which contains an active ingredient being a drug such as indomethacin and a hydrophobic polymer like PCL since fast release of indomethacin leads to saturation of indomethacin in water. This second mixture is obtained by mixing two solutions and making a blend: PCL in chloroform and then indomethacin solution in methanol. This blend is then slot die coated onto the first lid layer 602b into a thickness of 50-100 microns, step 707b, to form a core film layer 603b. The core film layer with the active ingredient may then be dried, which may be done in a short time by using infrared, IR, heating or under a hot furnace at 40-50° C. within one minute or more. The active ingredient can be heated in the slot die coater after coating to change the crystallinity of either the active ingredient or the polymer. For significantly changing the crystallinity of indomethacin, any temperatures >80° C. for 15-30 seconds is enough. The temperature is high enough to lead to crystallinity but low enough to not degrade the drug, 610b and step 708b. Alternatively, the active ingredient can also be coated without any polymer matrix by making the active ingredient slurry and coating it. The slurry will allow maximum loading of active ingredient in the micro punched particles.

The substrate 601b with the lid film layer 602b and the core film layer 603b is now fed to a third slot die coater 609b, step 709b. For the third layer a third mixture is provided, step 710b, which contains a biodegradable polymer like polycaprolactone, PCL, or polylactic acid, PLA, dissolved in a solvent, such as dichloromethane, DCM, choloroform, acetone, and methanol and then coated as liquid. The third mixture is fed to the third slot die coater 609b to be slot die coated onto the second layer to provide a wet shell film layer of a thickness of 10-100 μm, 604b and step 711b. The shell film layer may then be dried, which may be done in a short time by using infrared, IR, heating or under a hot furnace at 40-50° C. within one minute or more, step 712b. Alternatively polymer may be supplied in melted form to the third slot die coater 609b or melted within the slot die head of the slot die coater 609b, to be coated onto the second core layer. The melted polymer is dried instantaneously, when the polymer is deposited. It is noted that the deposited shell layer 604b is thicker than the lid layer 602b. As an example, the lid layer may be about 10 μm and the shell layer about 50 μm, while the core layer may be about 75 μm.

After the deposition of all the film layers, the deformable substrate 601b holding the lid film layer 602b, the active ingredient core film layer 603b and the shell layer 604b is fed to a punching station, 605b and step 713b. At the punching station 605b the film layers 602b, 603b, 604b are punched by a Ni Stamp having protrusions with a depth of 100-200 μm and a diameter of 50-500 μm either by plate to plate or roll to roll or roll to plate forms to obtain separate microstructures 605b on the substrate 601b, step 714b. For the above example of thicknesses of the film layer, the depth of the protrusions should be larger than 135 μm, such as about 150 μm.

The punching may be performed as a plate to plate punching at about 4 bars pressure for 5 minutes with heat supplied to ensure drawing of the shell layer around the core layer, and for shell layers based on PCL, heat is supplied during the punching to ensure the temperature can be kept at 60-70° C., while for PLA based shell layers, heat is supplied during the punching to ensure the temperature can be kept at 90-120° C. In another embodiment, the punching is performed as a roll to roll punching at a high pressure such as about or above 10 bars within a short time of about 1 minute and without heating during the period of high pressure. The high-pressure period may be followed by a short period of about 30 seconds for flash heating the lid layer to the shell layer, which has been punched and drawn around the core layer down to the lid layer. For PCL based shell layers, the flash heating should be about 60-70° C. and for PLA based shell layers, the flash heating should be about 90-120° C.

Finally, the substrate 601b holding the punched microstructures or microparticles 606b, may be fed to a separation station, step 715b, to be separated from the substrate 601b, step 716b. In some embodiments, then after the punching process the microstructures may be stuck into the protrusions of the punching stamp, and the separation of the microstructures 606a will include a separation of the microstructures from the protrusions of the stamp.

FIGS. 6c and 7c illustrate a third example 600c. The handling substrate 601c is made of a deformable material, which is a polyethylene sheet having thickness in the range of 100 μm to a few mms. The substrate 601c is fed to a first slot die coater 607c, step 701b.

For the first layer 602c a first mixture is provided, step 702c, which contains a biodegradable polymer like polycaprolactone, PCL, which may be dissolved in a solvent, such as dichloromethane, DCM. The first mixture is fed to the first slot die coater 607c to be slot die coated onto the substrate 601c to provide a wet lid film layer of a thickness of 2-50 μm, 602c and step 703c. The lid film layer may then be allowed to dry, which may be done in a short time by using infrared, IR, heating or under a hot furnace at 40-50° C. within one minute or more, step 704c. In another embodiment, then the biodegradable polymer may be supplied in melted form to the first slot die coater 607c, to be coated onto the substrate 601c. The melted polymer is dried instantaneously, when the polymer is deposited. This first film layer 602c acts as the lid film layer.

The substrate 601c with the lid film layer 602c is now fed to a second slot die coater 608c, step 705c. For the second layer, an active ingredient being a drug, such as furosemide is provided in a slurry form with acetone in a first syringe 610c. Furosemide does not degrade at high temperatures of up to 200° C. A polymer, such as PCL, is provided in melted form in a second syringe 611c, which is heated at 80° C. to melt the PCL. The slot die head of the second slot die coater 608c has two inlet holes connected to the two syringes 610c and 611c, whereby the slurry furosemide and the melted PCL can be supplied to the second slot die coater 608c, step 706c. The slot die head of the slot die coater 608c is heated at 80-100° C. to ensure that PCL is in melted form while the furosemide slurry comes in the slot die head and blends with melted PCL. The heated blend is now deposited on the first film layer 602c to form a core film layer 603c, step 707c. Given that acetone has very low boiling point, it would be almost completely evaporated as soon as the furosemide slurry-melted PCL blend would come out of the slot die head and be coated on the substrate 601c, where the melted polymer is dried almost instantaneously, when the polymer is deposited, step 708c.

The substrate 601c with the lid film layer 602c and the core film layer 603c is now fed to a third slot die coater 609c, step 709c. For the third layer a third mixture is provided, which contains a biodegradable polymer like polycaprolactone, PCL, which may be dissolved in a solvent, such as dichloromethane, DCM, step 710c. The third mixture is fed to the third slot die coater 609c to be slot die coated onto the core film layer 603c to provide a wet shell film layer of a thickness of 10-100 μm, 604c and step 711c. The lid film layer may then be allowed to dry, which may be done in a short time by using infrared, IR, heating or under a hot furnace at 40-50° C. within one minute or more, step 712c. In another embodiment, then the biodegradable polymer may be supplied in melted form to the third slot die coater 609c, to be coated onto the core film layer 603c. The melted polymer is dried instantaneously, when the polymer is deposited. This third film layer 604c acts as the shell film layer. As an example, the lid layer may be about 10 μm and the shell layer about 50 μm, while the core layer may be about 75 μm.

Again, after the deposition of all the film layers, the deformable substrate 601c holding the lid film layer 602c, the active ingredient core film layer 603c and the shell layer 604c is fed to a punching station, 605c and step 713c. At the punching station 605c the film layers 602c, 603c, 604c are punched by a Ni Stamp having protrusions with a depth of 100-200 μm and a diameter of 50-500 μm either by plate to plate or roll to roll or roll to plate forms to obtain separate microstructures 605c on the substrate 601c, step 714c. For the above example of thicknesses of the film layer, the depth of the protrusions should be larger than 135 μm, such as about 150 μm.

The punching may be performed as a plate to plate punching at about 4 bars pressure for 5 minutes with heat supplied to ensure drawing of the shell layer around the core layer, and for shell layers based on PCL, heat is supplied during the punching to ensure the temperature can be kept at 60-70° C. In another embodiment, the punching is performed as a roll to roll punching at a high pressure such as about or above 10 bars within a short time of about 1 minute and without heating during the period of high pressure. The high-pressure period may be followed by a short period of about 30 seconds for flash heating the lid layer to the shell layer, which has been punched and drawn around the core layer down to the lid layer. For PCL based shell layers, the flash heating should be about 60-70° C.

Finally, the substrate 601c holding the punched microstructures or microparticles 606c, may be fed to a separation station, step 715c, to be separated from the substrate 601c, step 716c. In some embodiments, then after the punching process the microstructures may be stuck into the protrusions of the punching stamp, and the separation of the microstructures 606c will include a separation of the microstructures from the protrusions of the stamp.

FIGS. 6d and 7d illustrate a fourth example 600d. The handling substrate 601d is made of a deformable material, which is a polyethylene sheet having thickness in the range of 100 μm to a few mms. The substrate 601d is fed to a first slot die coater 607d, step 701d.

For the first layer 602d a first mixture is provided, step 702d, which contains a biodegradable polymer like polycaprolactone, PCL, which may be dissolved in a solvent, such as dichloromethane, DCM. The first mixture is fed to the first slot die coater 607d to be slot die coated onto the substrate 601d to provide a wet lid film layer of a thickness of 2-50 μm, 602d and step 703d. The lid film layer may be allowed to dry, which may be done in a short time by using infrared, IR, heating or under a hot furnace at 40-50° C. within one minute or more, step 704d. In another embodiment, then the biodegradable polymer PCL may be supplied in melted form to the first slot die coater 607d, to be coated onto the substrate 601d. The melted polymer is dried instantaneously, when the polymer is deposited. This first film layer 602d acts as the lid film layer. This lid film layer may have a thickness of 5 μm.

The substrate 601d with the lid film layer 602d is now fed to a second slot die coater 608d, step 705d. For the second layer, an active ingredient being a drug, such as furosemide is provided in a slurry form with acetone. The slurry furosemide is supplied to the second slot die coater 608d, step 706d, and deposited on the first film layer 602d to form a core film layer 603d, step 707d. The core film layer 603d may be allowed to dry, which may be done in a short time by using infrared, IR, heating or under a hot furnace at 40-50° C. within one minute or more, step 708d. The core film layer may have a thickness of 100 μm.

The substrate 601d with the lid film layer 602d and the core film layer 603d is now fed to a third slot die coater 609d, step 709d. For the third layer a third mixture is provided, which contains the same biodegradable polymer as the lid film layer, such as polycaprolactone, PCL, which may be dissolved in a solvent, such as dichloromethane, DCM, step 710d. The third mixture is fed to the third slot die coater 609d to be slot die coated onto the core film layer 603d to provide a wet shell film layer of a thickness of 50-80 μm, 604d and step 711d. The lid film layer may then be allowed to dry, which may be done in a short time by using infrared, IR, heating or under a hot furnace at 40-50° C. within one minute or more, step 712d. In another embodiment, then the biodegradable polymer may be supplied in melted form to the third slot die coater 609d, to be coated onto the core film layer 603d. The melted polymer is dried instantaneously, when the polymer is deposited. This third film layer 604d acts as the shell film layer. In this example, for which the lid layer 602d and the shell layer 604d are of the same material, the lid layer is about 5 μm and the shell layer about 70 μm, while the core layer is about 100 μm.

Again, after the deposition of all the film layers, the deformable substrate 601d holding the lid film layer 602d, the active ingredient core film layer 603d and the shell layer 604d is fed to a punching station, 605d and step 713d. At the punching station 605d the film layers 602d, 603d, 604d are punched by a Ni Stamp having protrusions with a depth of 100-200 μm and a diameter of 50-500 μm either by plate to plate or roll to roll or roll to plate forms to obtain separate microstructures 605d on the substrate 601d, step 714d. For the above example of thicknesses of the film layer, the depth of the protrusions should be larger than 175 μm, such as about 190 μm.

The punching may be performed as a plate to plate punching at about 4 bars pressure for 5 minutes with heat supplied to ensure drawing of the shell layer around the core layer, and for shell layers based on PCL, heat is supplied during the punching to ensure the temperature can be kept at 60-70° C. In another embodiment, the punching is performed as a roll to roll punching at a high pressure such as about or above 10 bars within a short time of about 1 minute and without heating during the period of high pressure. The high-pressure period may be followed by a short period of about 30 seconds for flash heating the lid layer to the shell layer, which has been punched and drawn around the core layer down to the lid layer. For PCL based shell layers, the flash heating should be about 60-70° C.

Finally, the substrate 601d holding the punched microstructures or microparticles 606d, may be fed to a separation station, step 715d, to be separated from the substrate 601d, step 716d. In some embodiments, then after the punching process the microstructures may be stuck into the protrusions of the punching stamp, and the separation of the microstructures 606d will include a separation of the microstructures from the protrusions of the stamp.

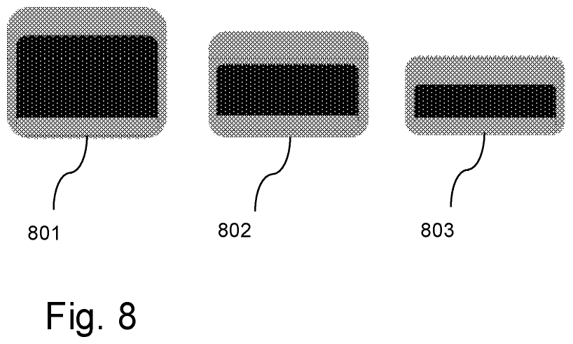

FIG. 8 is a schematic illustration of three 3-layered microstructures 801, 802, 803 with different thicknesses of a middle layer holding an active ingredient according to example embodiments. The microstructures 801, 802 and 803 may be manufactured as described in the second example and illustrated by FIGS. 6b and 7b. Here, the PCL is used both for the lid layer and the shell layer, while the core layer is made from a blend of PCL in chloroform and a solution of the active ingredient indomethacin in methanol. For all three microstructures 801, 802 and 803, the lid layer has a thickness of about 5 μm and the shell layer has a thickness of about 50 μm, while the thickness of the core layer varies. For the structure 801, the core layer has a thickness about 150 μm, for the structure 802, the core layer has a thickness about 100 μm, and for the structure 803, the core layer has a thickness about 50 μm. Based on the thickness of the core layer holding the active drug ingredient, the time of drug release is controlled. Other parameters for drug release like crystallinity of the drug, which may be based on the core layer thickness, can also be altered by applying heat during the punching process. Thinner core layer means less room and material for the drug particles to wiggle around and clump together to grow crystals. This could mean less crystallinity in thin films, which implies that there may be faster drug release from the thinner films.

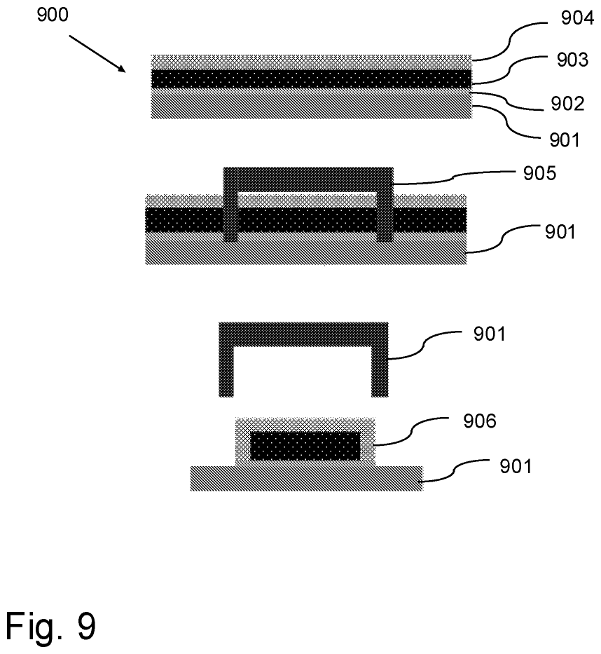

FIG. 9 is a schematic illustration of punching and harvesting of microstructures 906 stored on a handling substrate 901 according to an example embodiment. FIG. 9 shows a handling substrate, which is made of a deformable material, or has a deformable layer 901, which is chosen such that the material for the layer has inherent high surface energy or the layer is surface treated to increase the surface energy. The deformable layer 901 of the substrate is coated with a lid film layer 902 being the first layer, which is coated with a core film layer 903 being the second layer, which may hold an active ingredient, with the core film layer 903 coated with a shell layer 904, which is the third layer. A rigid stamp 905 having protrusions with sidewalls is used for punching, with the protrusions of the stamp 905 penetrating fully through the three film layers 902, 903 and 904 to reach the deformable substrate 901. The high surface energy of the deformable layer 901 leads to the adhesion of the punched microstructures or particles 906 to the deformable layer 901 instead of the protrusions of the stamp 905, which is itself coated with a Teflon non-sticky coating.

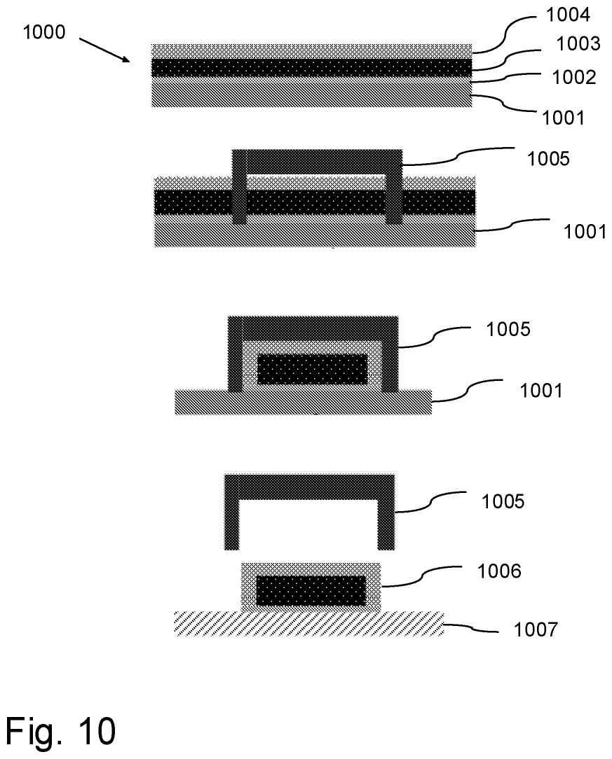

FIG. 10 is a schematic illustration of punching and harvesting of microstructures stored in protrusions of a punching stamp according to an example embodiment. FIG. 10 shows a handling substrate, which is made of a deformable material, or has a deformable layer 1001. The deformable layer 1001 of the substrate is coated with a lid film layer 1002 being the first layer, which is coated with a core film layer 1003 being the second layer, which may hold an active ingredient, with the core film layer 1003 coated with a shell layer 1004, which is the third layer. A rigid stamp 1005 having protrusions with sidewalls is used for punching, with the protrusions of the stamp 1005 penetrating fully through the three film layers 1002, 1003 and 1004 to reach the deformable substrate 1001. In this case, the deformable layer 1001 has a low surface energy or the protrusions of the stamp 1005 are not coated with any anti-stiction coating. The large contact surface area of the polymer with the protrusions of the stamp 1005 leads to higher adhesion of the microstructures 1006 to the protrusions resulting in sticking of the microstructures 1006 to the protrusions of the stamp 1006 after punching. This requires another step to release the punched microstructures 1006 particles by bonding them to another high surface energy release layer 1007. This bonding step can be as simple as taking a tape and peeling it off the stamp 1005 to extract all the microstructures 1006 attached to the stamp 1005, or it can be a heating step to make the microstructures 1006 adhere to a release layer. Other methods can be UV bonding or welding. The microstructures or particles 1006 may be bonded to a PAA-PEG release layer at 100° C. for 1 min under 1.9 MPa pressure, cooled down to room temperature and demolded. Other bonding layer materials can be other water soluble or hydrophilic polymers like poly vinyl alcohol or pH sensitive layers like Eudragit or other “sticky” materials with inherently high surface energy.

Rolling-Up for Storage after Slot Die Coating

It is within embodiments that punching of the manufactured film structure may be done at a later stage. Also, the addition of an extra film layer by slot die coating may be done at a later stage. There can be a rolling-up of a film structure for storage after any slot die coating process, e.g. when only one slot die coating station is part of the production line and multiple film layers need to be coated. After the final slot die coating process, the punching step could be directly inline, that is with no final roll-up, or from unwinding a completed product, where all slot die coating processes have been performed.

Rigid Stamp

The rigid stamp can be made of any plastic material or metal or thermosets as long as the material is robust under the applied pressure during the punching processing, which pressure may be equal to or higher than 10 bars. Also, it is preferred that the rigid stamp is made of a material having a higher stiffness than the deformable layer of the handling substrate, which implies that the deformable layer is susceptible to deformation by the rigid stamp. Examples of material, which are robust under the applied pressures are Ni stamps, stainless steel, and tungsten.

Protective Shell Layer and Lid Layer

For oral drug delivery applications, the shell layer and the lid layer should be made of quick dissolving materials that dissolve in the media as soon as the particles enter the body. The shell and lid layers may be made of: biodegradable and biocompatible thermoplastics, thermosets like hydrogels, waxes, paraffins, collagen, or Ph sensitive polymers like Eudragit. The shell and lid layers can be made of the same material but with different thicknesses so that they degrade at different times in the human system. Therefore, precise control in the uniformity and thickness of such films is of great importance. Such precise control can be obtained by slot die coating of the films.

Examples of preferred materials for shell layer and lid layer: polycaprolactone (PCL), polylactic acid (PLA), polyglycolic acid (PGA), hydroxypropylmethyl cellulose (HPMC), polymethacrylate (PMMA), Eudragits (Poly(methacylic acid-co-methyl methacrylate), ethyl cellulose (EC), polyethylene glycol methacrylate (PEGMA), polyethylene glycol dimethacrylate (PEGDMA), poly(lactic-co-glycolic acid) (PGLA), polyacrylic acid (PAA), or co-polymers of at least one of the above polymers, materials that turn liquid when slightly heated and can become solid at room temperature like: wax, oils, paraffins, butter and other oil-based, fat-based materials, hydrogels. The melting of polymers may be done from 37° C. to 200° C.

Core Layer

The core layer may hold an active ingredient, which may be selected from the list: small organic molecules, proteins, peptides, vitamins, minerals antibodies, antibody fragments, vaccines, RNA, DNA, antibiotics or combinations, screening materials, diagnostic materials like certain metals, and enzymes.

For preparing the core layer, the active ingredient may be deposited as a slurry in a fast evaporating solvent, which leaves the core layer as a thin powdered layer. The core layer may also hold a drug matrix made of a polymer and an active ingredient. The polymer can be made of a polymer material and chosen from the above list for polymer material for the lid and shell layers, but the polymer can also be water soluble like PAA as described in the examples. The polymer can also be a mucoadhesive polymer like chitosan.

Deformable Layer of Handling Substrate

It is within embodiments that the handling substrate may hold a deformable top surface layer, which may be provided by slot die coating. The deformable layer may be made of a thermoplastic film, thermosets like polydimethylsiloxane (PDMS), silicones, rubbers, and even metals like aluminum sheets. The main requirement for choosing the material for the deformable layer is that it should be softer than the rigid stamp material in order to not destroy the protrusions on the stamp. The deformable layer may also be a tape or made of a water-soluble material

Barrier Layer or Release Controlling Layer

It is within embodiments that the multi-layered film structure may hold one or more barrier layers. A barrier layer may be placed between two core layers with active ingredients to allow control of the release time of active ingredient. These barrier layers should be stable under the release conditions. They can be of biodegradable and biocompatible materials as stated above for the shell and lid layers. As an example, a multilayered microstructure may be designed for a pulsatile release by having several core layers separated by release layers, see FIG. 16.

Mucoadhesive Layers

The lid film layer can be made of a mucoadhesive. Functional mucoadhesive coatings can be placed at the walls of the shell or the entire polymer matrix of the core layer, in which the active ingredient is embedded can be mucoadhesive. The function of the mucoadhesive layer is to make the microstructures sticky especially on the side where the active ingredient comes out. This may ensure tight attachment of the microstructures with the bodily mucosa. Because of this attachment, the microstructures or particles will not just flow and be flushed out of the system, whereby they will be available for longer times to deliver the active ingredient in a slow fashion increasing the bioavailability of the microstructures or particles. Mucoadhesion can be achieved by using special hydrophilic layers like PAA, PVP, or Chitosan, or by having special topography or flat shape of the particles.

Film Layer Thickness

As can be seen from the above described examples, the thickness of the different film layers may vary from as low as 5 μm to as high as 200 μm.

Width and Length of Handling Substrate

The width of the handling substrate may depend on the dimensions of the rigid stamp used for punching. For the Ni stamp described above, the handling substrate may have a width of 6 inches. However, when using slot die coating, the slot die can coat a uniform stripe from a width of a few mm up to several meters, and the substrate can equally be from a few mm to several meters width. The slot die can coat any width up to the total width of the substrate and is not constrained by half or ⅔ of the substrate. The slot die can also be constructed to coat multiple stripes in parallel in the direction of coating, such as for example 20×10 mm wide stripes with 5 mm spacing between stripes on a 305 mm wide substrate. Substrate widths are typically from a few cm to several meters. For the slot die coating length, the coating length could be from cm to km, only constrained by a continuously available substrate length.

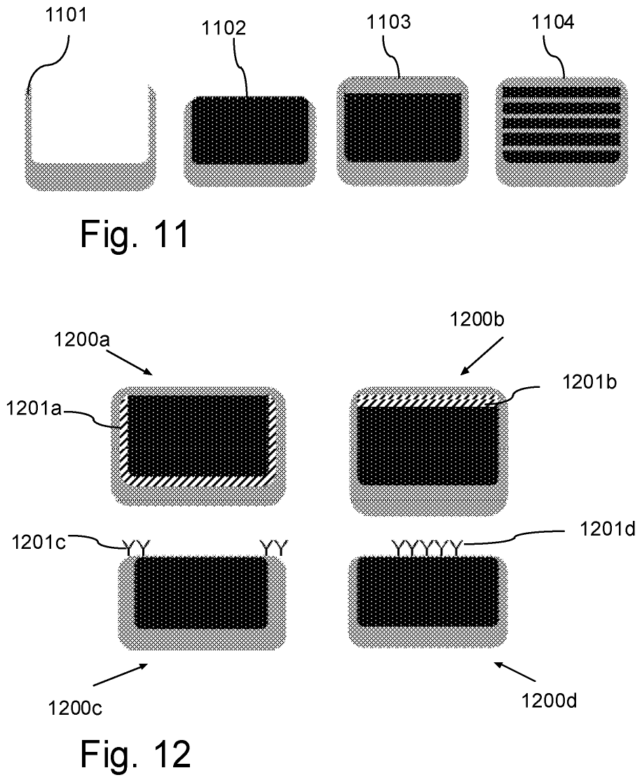

FIG. 11 is a schematic illustration of microstructures 1101, 1102, 1103, 1104 with different number of film layers holding an active ingredient according to example embodiments. The structure 1101 shows the shell of an empty microstructure, structure 1102 shows a microstructure made up of a shell and a core layer without any lid layer. For the structure 1102 the core layer may be a mucoadhesive polymer matrix in which the active ingredient is embedded, which may ensure tight attachment of the microstructure 1102 with the bodily mucosa. The structure 1103 shows a microstructure having a shell, a core and a lid layer. Also here, the core layer may be a mucoadhesive polymer matrix in which the active ingredient is embedded, but here the lid layer needs to be dissolved before the active ingredient can be released. The structure 1104 shows a multi-layered microstructure having a shell, a first core layer, a first release layer, a second core layer, a second release layer, a third core layer, a third release layer, a fourth core layer, a fourth release layer, a fifth core layer and a lid layer. For the structure 1104 the lid layer and the release layer may be made of the same dissolving material, whereby a pulsating release of the active ingredient from the different core layers can be obtained, see FIG. 16.

FIG. 12 is a schematic illustration of different microstructures 1200a, 1200b, 1200c, 1200d provided with a mucoadhesive material according to example embodiments. The microstructure 1200a has a functional mucoadhesive coating 1201a placed between the core layer and the shell layer. The microstructure 1200b has a functional mucoadhesive coating 1201b placed between the lid layer and the core layer. The microstructure 1200c has a core layer and a shell layer but no lid layer and further has functional mucoadhesive particles 1201c placed on shell end walls facing the release side of the structure 1200c. The microstructure 1200d has a core layer and a shell layer but no lid layer and further has functional mucoadhesive particles 1201d placed on the side of the core layer facing the release side of the structure 1200d.

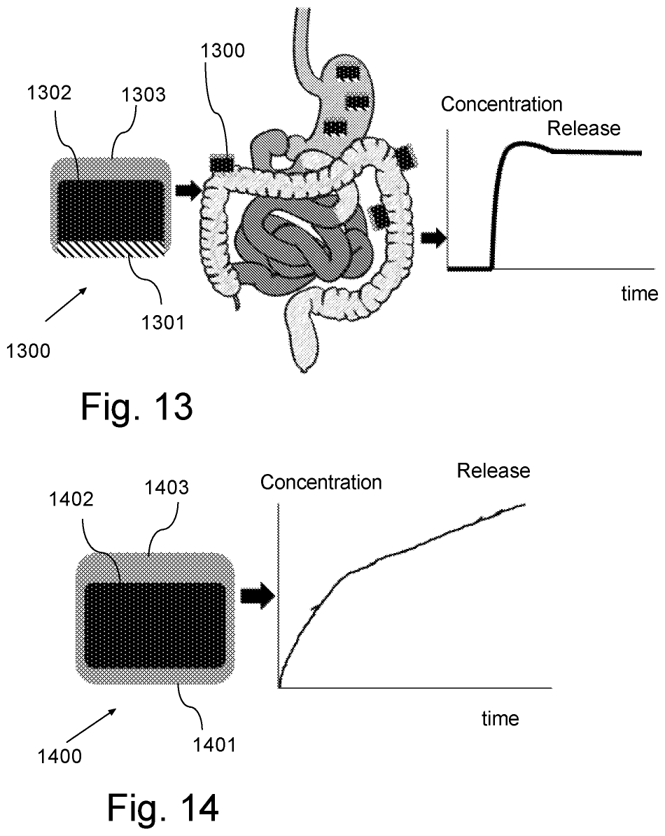

FIG. 13 is a schematic illustration of a microstructure 1300 manufactured for a targeted release of an active ingredient according to an example embodiment. The microstructure 1300 has a lid layer 1301 made of Eudragit, a core layer 1302 holding the drug budesonide as the active ingredient, and a shell layer 1303 made of PCL. Due to the presence of the Eudragit layer 1301, the drug can be targeted in the intestine for the localized treatment of inflammatory bowel disease, IBD. The Eudragit lid dissolves in the intestine releasing budesonide from the microparticles locally.

The microstructures 1300 may be manufactured by use of slot die coating and punching processes according to an embodiment. A handling substrate is provided, which substrate is of a deformable material, which is a polyethylene sheet having thickness in the range of 100 μm to a few mms. The substrate is fed to a first slot die coater. For the first layer a first mixture is provided, which contains Eudragit dissolved in isopropanol, IPA. The first mixture is fed to the first slot die coater to be slot die coated onto the substrate to provide a wet lid film layer of a thickness of 10-20 μm. The lid film layer is then allowed to dry, which may be done in a short time by using infrared, IR, heating or under a hot furnace at 40-50° C. within one minute or more, such as within 5-10 minutes. This first Eudragit layer acts as the lid layer of the microparticles 1300.

The substrate with the lid film layer is now fed to a second slot die coater. For the second layer a second mixture is provided, which contains an active ingredient being a drug such as budesonide and a hydrophobic polymer like soluplus. This second mixture is obtained by mixing two solutions and making a blend: Soloplus and budesonide solutions in DCM. This blend is then slot die coated onto the first lid layer into a thickness of 100 microns to form a core film layer. The core film layer with the active ingredient may then be dried, which may be done in a short time by using infrared, IR, heating or under a hot furnace at 40-50° C. within one minute or more.

The substrate with the lid film layer 1301 and the core film layer 1302 is now fed to a third slot die coater. For the third layer a third mixture is provided, which contains a biodegradable polymer like polycaprolactone, PCL, dissolved in a solvent, such as dichloromethane, DCM, chloroform, acetone, and methanol and then coated as liquid. The third mixture is fed to the third slot die coater to be slot die coated onto the second layer to provide a wet shell film layer of a thickness of 50-80 μm. The shell film layer may then be allowed to dry, which may be done in a short time by using infrared, IR, heating or under a hot furnace at 40-50° C. within one minute or more, step 712b. Alternatively polymer may be supplied in melted form to the third slot die coater or melted within the slot die head of the slot die coater 609b, to be coated onto the second core layer. The melted polymer is dried instantaneously, when the polymer is deposited. It is noted that the deposited shell layer 604b is thicker than the lid layer 602b.

After the deposition of all the film layers, the deformable substrate holding the lid film layer 1301, the active ingredient core film layer 1302 and the shell layer 1303 is fed to a punching station for being punched and separated into microparticles 1300. The punching and separation may be performed as described in connections with the examples of FIGS. 6a-6d.

FIG. 14 is a schematic illustration of a microstructure 1400 manufactured for a sustained release of an active ingredient according to an example embodiment. The microstructure 1400 has a lid layer 1301 made of Eudragit, a core layer 1402 holding the drug indomethacin as the active ingredient, and a shell layer 1403 made of PCL.

The microstructures 1400 may be manufactured in almost the same way as the microstructures 1300 of FIG. 13, with the first lid layer 1401 and the third shell layer 1403 being prepared in the same way as described for the structure 1300.

For the second core layer 1402 a second mixture is provided, which contains an active ingredient being a drug such as indomethacin and a hydrophobic polymer like PCL. This second mixture is obtained by mixing two solutions and making a blend: PCL in chloroform and indomethacin in methanol. This blend is then slot die coated onto the first lid layer into a thickness of 50-100 microns to form a core film layer. The core film layer with the active ingredient may then be allowed to dry, which may be done in a short time by using infrared, IR, heating or under a hot furnace at 40-50° C. within one minute or more. The punching and separation may also be performed as described in connections with the examples of FIGS. 6a-6d.

Due to the embedding of the active ingredient indomethacin in a hydrophobic, slow degrading polymer PCL, the release of 100% indomethacin will take few hours to days in the intestine once Eudragit lid layer dissolves in the intestinal media. By controlling the shape of the microparticles, e.g.: by making them flat, better adhesion of the microparticles to the intestinal mucosa can be ensured allowing for longer drug release and absorption periods.

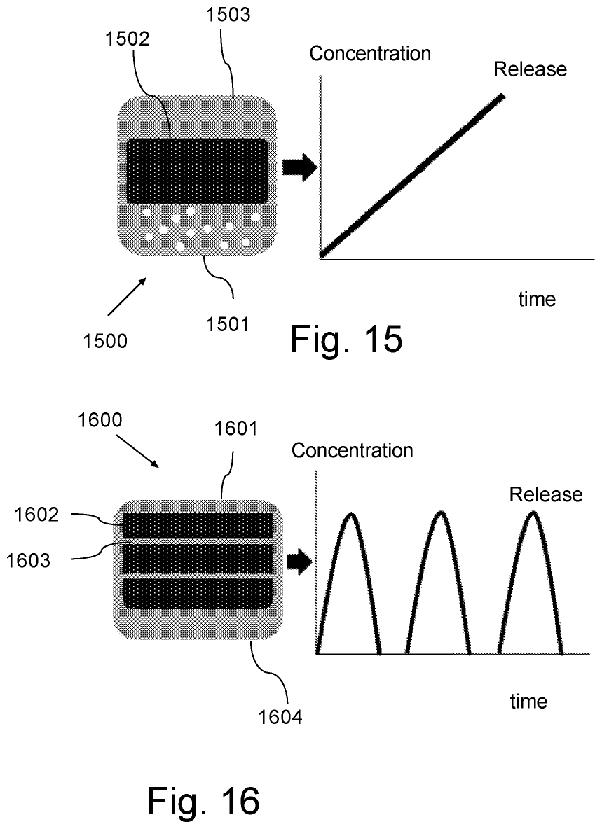

FIG. 15 is a schematic illustration of a microstructure 1500 manufactured for a controlled release of an active ingredient according to an example embodiment. The microstructure 1500 has a porous lid layer 1501, a core layer 1502 holding the drug budesonid as the active ingredient, and a shell layer 1503 made of PCL.

For the first lid layer 1501 a porous film layer of PLA or PLGA is fabricated to a thickness of 50 μm using the process in the example described in connection with FIGS. 6a and 7a. This layer is oxygen plasma treated for 30 sec to introduce hydrophilicity to the layer for the coating of next active ingredient containing layer 1502.

For the second layer 1502, a mixture is used holding a small molecule drug such as budesonide and a hydrophilic polymer such as soluplus. This layer can be prepared to a thickness of about 100 μm as mentioned in the example on targeted delivery as described in connection with FIG. 13.

The third shell layer 1503 is also prepared in the same way as described for the third layer 1303 of the structure 1300 of FIG. 13. Thus, for the third shell layer, PCL is used, which is slot die coated onto the second layer into a thickness of 50-80 microns. This is coated either as solution on top of the second layer 1502 or as a melted liquid with the parameters described before. This third topmost layer 1503 act as the protective shell layer. The punching and separation may also be performed as described in connections with the examples of FIGS. 6a-6d.

Due to the presence of a lid 1501 with small holes, budesonide, which has lot of side effects if taken above the dosage required, is released in a zero-order controlled release profile. This means that at no point of the release, a lot of budesonide is dumped in the system. The lid 1501 with the holes is intact during the whole period of drug release controlling the release rate. That is why the lid 1501 is chosen to be made up of PLA/PLGA instead of a pH sensitive polymer.

FIG. 16 is a schematic illustration of a microstructure 1600 manufactured for a pulsatile release of an active ingredient according to an example embodiment. The microstructure 1600 has a lid layer 1601 holding Eudragit, three core layers 1602 holding the drug insulin as the active ingredient, two barrier layers 1603 made of PLGA, and a shell layer 1603 made of PCL.

When manufacturing the microstructure 1600, a first lid layer holding Eudragit is coated to a thickness of 10-20 μm on a handling substrate as described in connection with the microstructure 1300 of FIG. 13.

For the second layer, which is also the first core layer, 1603, a mixture of insulin dissolved in an aqueous solution of water-soluble polymer like PAA is used. This mixture is slot die coated onto the first layer 1601 into a thickness of 10-20 μm. This core film layer with insulin is then allowed to dry, which may be under infrared, IR, heating or under a hot furnace at 40-50° C. within one minute or more.

For the third layer, which is also the first barrier layer, PLGA is slot die coated onto the first core layer into a thickness of 5-10 microns. This PLGA layer is coated either as a solution or melted polymer on top of the first core layer. Both the presence of organic solvent in the PLGA solution and the high temperature treatment of the melted PLGA can in principle destroy the active ingredient in the first core layer to certain degree. Assuming that this degradation is only confined to the topmost layer of first core layer that comes in contact with the PLGA barrier layer, the first barrier layer can be slot die coated directly on the first core layer. Otherwise, a PLGA barrier layer can be coated on another handling substrate and then assembled on the Al layer later on. This would increase the numbers of processing steps and hence processing time. PLGA is used as a barrier layer as it can degrade in the bodily fluids.

For the fourth layer, which is the second core layer, another insulin containing layer is coated on the first PLGA barrier layer. On top of this fourth layer, a fifth layer, which is a second PLGA barrier layer is coated, and on top of this second barrier layer is coated a sixth layer, which is a third core insulin layer. This would generate 3 pulses of insulin delivery.

For the last seventh layer, which is the shell layer 1604, PCL is used, which is slot die coated onto the second layer into a thickness of 50-80 microns. This is coated either as solution on top of the third core layer or as a melted liquid with the parameters, again taking into the consideration, the effect of such processing on the fragile insulin cargo. The punching and separation may be performed as described in connections with the examples of FIGS. 6a-6d.

Due to the presence of the barrier PLGA layers 1603, the insulin is released in pulses. This is beneficial for the case of insulin, as insulin spikes after every mealtime. By choosing the right thicknesses of the barrier layers, the times when insulin is released can be tallied with the three general mealtimes, reducing the frequency of administration of insulin, resulting in better patient compliance.

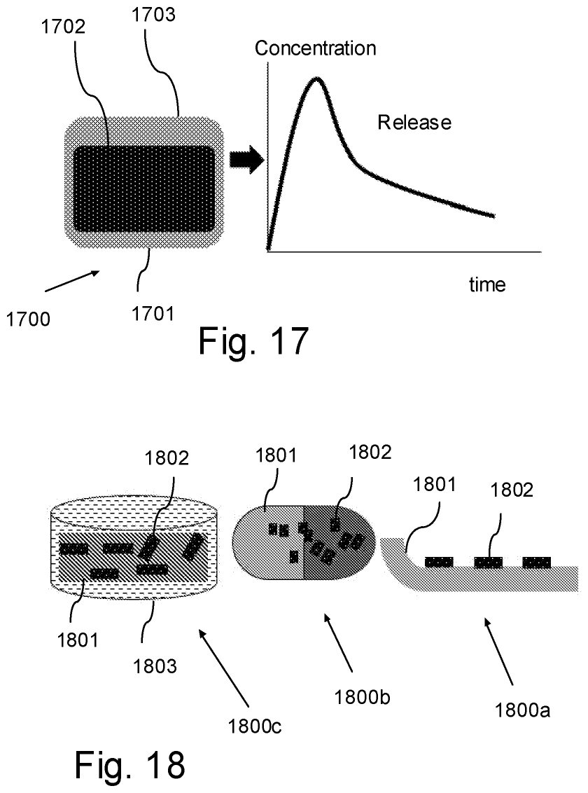

FIG. 17 is a schematic illustration of a microstructure 1700 manufactured for a burst release of an active ingredient according to an example embodiment. The microstructure 1700 has a lid layer 1701 holding Eudragit, a core layer 1702 holding a pain killer like ibuprofen as the active ingredient, and a shell layer 1703 made of PCL.

When manufacturing the microstructure 1700, a first lid layer holding Eudragit is coated to a thickness of 10-20 μm on a handling substrate as described in connection with the microstructure 1300 of FIG. 13. For the second layer being the core layer 1702, the pain killer like ibuprofen is provided in slurry form and slot die coated on the lid layer 1701, where after the slot die coated film is allowed to dry. The thickness of the core layer 1702 may be 50-100 μm.

For the third layer, which is the shell layer 1703, PCL is used, which is slot die coated onto the second layer into a thickness of 50-80 microns. This is coated either as solution on top of the third core layer or as a melted liquid with the parameters, again taking into the consideration, the effect of such processing on the fragile insulin cargo. The punching and separation may be performed as described in connections with the examples of FIGS. 6a-6d.

Researchers seek to avoid burst release, because the initial high release rates may lead to drug concentrations near or above the toxic level in vivo. However, when combined with targeted delivery, burst release may be desired at the targeted site, after the coating has served its purpose of protecting the drug.

FIG. 18 is a schematic illustration of harvesting of microstructures 1802 from a water-soluble layer 1801 according to an example embodiment. A film of a water-soluble layer 1801 holding microstructures 1802 is shown at 1800a, at 1800b the microstructures 1802 are scraped from the film 1801, and at 1800c a piece of the film 1801 is cut and put into a cup of water, where the film 1801 is dissolved and the microstructures 1802 separated from the film 1801.

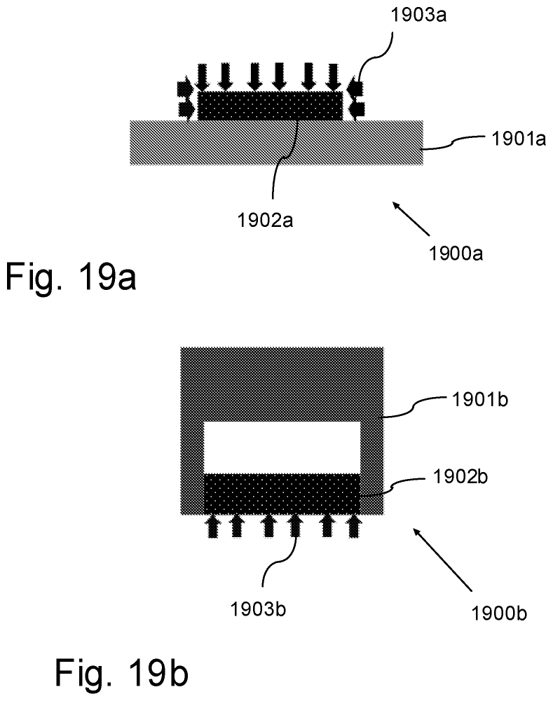

FIGS. 19a and 19b are schematic illustrations of surface treatment 1900a, 1900b of microstructures 1902a, 1902b according to example embodiments. FIG. 19a illustrates how microstructures 1902a, which are punched and are lying on a layer or substrate 1901a, can be coated 1903a with functional coatings like binders to other molecules or surface treated with plasma to change surface properties. FIG. 19b illustrates how microparticles 1903b, which are stuck in the protrusions of a stamp 1901b can be coated or surface treated 1903b before a bonding step for release of the microstructures 1902b from the protrusions 1901b.

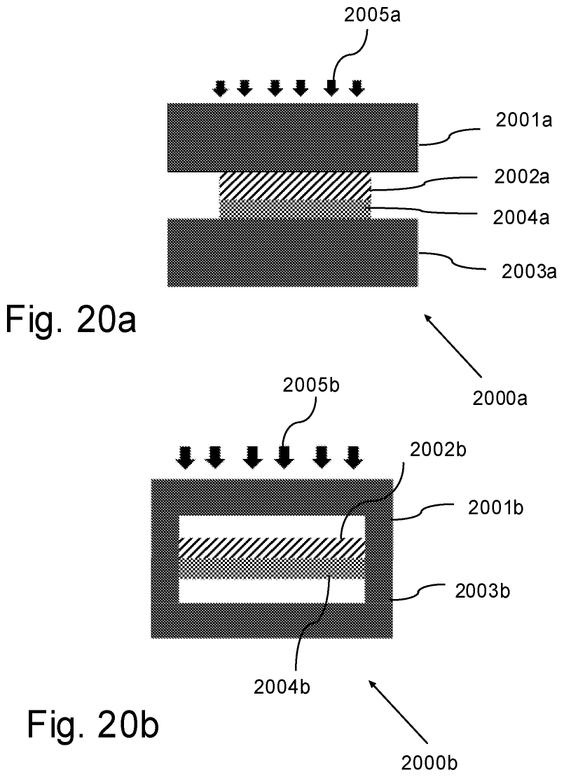

FIGS. 20a and 20b are schematic illustrations of bonding or fusion of two microstructures according to example embodiments. FIG. 20a shows a first microstructure 2002a, which has been punched onto a first layer or substrate 2001a and a second microstructure 2004a, which has been punched onto a second layer or substrate 2003a. The two microstructures 2002a and 2004a are aligned using X-Y alignment stage and bonded 2005a thermally by flash heating or other methods.

FIG. 20b shows a first microstructure 2002b, which has been punched and is stuck into a first protrusion 2001b of a stamp, and a second microstructure 2004b, which has been punched and is stuck into a second protrusion 2003b of a stamp. The two microstructures 2002b and 2004b are aligned using X-Y alignment stage and bonded 2005b thermally by flash heating or other methods. By bonding two microparticles 2002a, 2004a or 2002b, 2004b it is possible to obtain microstructures with many layers. Thus, it may be possible to give 8 pulses of insulin when fusing microstructures with 4 layers of insulin each.

The aspects of the disclosed embodiments have been described in conjunction with various embodiments herein. However, other variations to the disclosed embodiments can be understood and effected by those skilled in the art in practicing the claimed present disclosure, from a study of the drawings, the disclosure, and the appended claims. In the claims, the word “comprising” does not exclude other elements or steps, and the indefinite article “a” or “an” does not exclude a plurality.

1-117. (canceled)

118-184. (canceled)

185-237. (canceled)

238. A microstructure for delivery of an active ingredient to a user, said microstructure being produced in accordance with a method for manufacturing a multi-layered film structure, said method comprising:
a) providing a handling substrate having a length and a width, which handling substrate is of a deformable material having a deformable top surface, or which handling substrate holds a deformable top surface layer;
b) providing a plurality of film layers on top of the deformable surface of the handling substrate, said plurality of film layers comprising a core film layer holding an active ingredient; wherein
one or more of the film layers are provided by a slot die coating process, said slot die coating process of a film layer comprising:
supplying one or more materials for forming the film layer to a slot die head; and
dispensing the supplied materials in a liquid form from the slot die head while moving the slot die head relative to the handling substrate, said dispensed liquid form material being deposited as a wet film layer on top of the handling substrate, or on top of the deformable top surface layer, or on top of one or more previously provided film layers;
wherein the method for manufacturing the multi-layered film structure further comprises:
c) subjecting the plurality of film layers to a punching step for generation of a plurality of microstructures, said punching being performed by use of a rigid stamp having a plurality of protrusions defining a plurality of cavities, wherein the depth of the cavities is larger than the thickness of the plurality of film layers, and lower than the combined thickness of the plurality of film layers and the deformable surface part of the handling substrate, thereby allowing the protrusions of the rigid stamp to penetrate all the way through the plurality of film layers during punching to divide the plurality of film layers into a plurality of separated microstructures.

239. The microstructure according to claim 238, wherein the step of providing a plurality of film layers or providing one or more film layers on top of the deformable surface of the handling substrate comprises:
providing several film layers on top of the deformable surface of the handling substrate, said film layers comprising at least the following sequence of layers on top of the deformable surface of the handling substrate:
i) a lid film layer;
ii) a core film layer; and
iii) a shell film layer.

240. The microstructure according to claim 238, wherein a plurality of film layers are provided on top of each other by a plurality of said slot die coating processes subsequently following each other.

241. The microstructure according to claim 238, wherein at least one or all of said slot die coating processes further comprises heating or drying of the deposited wet film layer to obtain a solid film layer.

242. The microstructure according to claim 241, wherein at least one of said slot die coating processes further comprises application of pressure to the obtained solid film layer, such as exposing the solid film layer together with the handling substrate and any previously provided film layers to a calendaring process.

243. The microstructure according to claim 238, wherein the handling substrate holds a deformable top surface layer, said deformable top surface layer being provided by a slot die coating process.

244. The microstructure according to claim 238, wherein each of said provided film layers are provided by one of said slot die coating processes.

245. The microstructure according to claim 238, wherein the separated microstructures are separated from the deformable surface layer.

246. The microstructure according to claim 245, wherein the microstructures hold an outer shell layer, and wherein the microstructures are separated from the deformable surface layer by bonding the outer shell layer of the microstructures onto a release layer.

247. The microstructure according to claim 245, wherein microstructures hold an outer shell layer and a lid layer, wherein the microstructures are separated from the deformable surface layer by being attached within the protrusions of the stamp, and wherein the lid layer of the microstructures is bonded to a release layer.

248. The microstructure according to claim 246, wherein the release layer is selected from the list consisting of: tape, water soluble polymer layers.

249. The microstructure according to claim 245, wherein the handling substrate holds a deformable surface layer being made of a water-soluble material, and the microstructures are separated from the deformable surface layer by dissolving the deformable surface layer.

250. A method for manufacturing a multi-layered film structure, said method comprising:
a) providing a handling substrate having a length and a width, which handling substrate is of a deformable material having a deformable top surface, or which handling substrate holds a deformable top surface layer;
b) providing a plurality of film layers on top of the deformable surface of the handling substrate; wherein
one or more of the film layers are provided by a slot die coating process, said slot die coating process of a film layer comprising:
supplying one or more materials for forming the film layer to a slot die head; and
dispensing the supplied materials in a liquid form from the slot die head while moving the slot die head relative to the handling substrate, said dispensed liquid form material being deposited as a wet film layer on top of the handling substrate, or on top of the deformable top surface layer, or on top of one or more previously provided film layers;
wherein the method for manufacturing a multi-layered structure further comprises:
c) subjecting the plurality of film layers to a punching step for generation of a plurality of microstructures, said punching being performed by use of a rigid stamp having a plurality of protrusions defining a plurality of cavities, wherein the depth of the cavities is larger than the thickness of the plurality of film layers, and lower than the combined thickness of the plurality of film layers and the deformable surface part of the handling substrate, thereby allowing the protrusions of the rigid stamp to penetrate all the way through the plurality of film layers during punching to divide the plurality of film layers into a plurality of separated microstructures; and
wherein the microstructures hold an outer shell layer and a lid layer, the microstructures are separated from the deformable surface layer by being attached within the protrusions of the stamp, and the lid layer of the microstructures is bonded to a release layer.

251. A method for manufacturing a multi-layered film structure, said method comprising:
a) providing a handling substrate having a length and a width, which handling substrate is of a deformable material having a deformable top surface, or which handling substrate holds a deformable top surface layer;
b) providing a plurality of film layers on top of the deformable surface of the handling substrate; wherein
one or more of the film layers are provided by a slot die coating process, said slot die coating process of a film layer comprising:
supplying one or more materials for forming the film layer to a slot die head; and
dispensing the supplied materials in a liquid form from the slot die head while moving the slot die head relative to the handling substrate, said dispensed liquid form material being deposited as a wet film layer on top of the handling substrate, or on top of the deformable top surface layer, or on top of one or more previously provided film layers;
wherein the method for manufacturing a multi-layered structure further comprises:
c) subjecting the plurality of film layers to a punching step for generation of a plurality of microstructures, said punching being performed by use of a rigid stamp having a plurality of protrusions defining a plurality of cavities, wherein the depth of the cavities is larger than the thickness of the plurality of film layers, and lower than the combined thickness of the plurality of film layers and the deformable surface part of the handling substrate, thereby allowing the protrusions of the rigid stamp to penetrate all the way through the plurality of film layers during punching to divide the plurality of film layers into a plurality of separated microstructures; and
wherein the microstructures hold an outer shell layer, and the microstructures are separated from the deformable surface layer by bonding the outer shell layer of the microstructures onto a release layer.

252. The method according to claim 250, wherein the release layer is selected from the list consisting of: tape, water soluble polymer layers.

253. The method according to claim 250, wherein the protrusions of the rigid stamp are made of a material having a hardness being higher than the hardness of the deformable material or deformable surface layer of the handling substrate.

254. The method according to claim 250, wherein a plurality of film layers are provided on top of each other by a plurality of said slot die coating processes subsequently following each other.

255. The method according to claim 250, wherein at least one or all of said slot die coating processes further comprises heating or drying of the deposited wet film layer to obtain a solid film layer.

256. The method according to claim 255, wherein at least one of said slot die coating processes further comprises application of pressure to the obtained solid film layer, such as exposing the solid film layer together with the handling substrate and any previously provided film layers to a calendaring process.

257. The method according to claim 250, further comprising:
c) subjecting the plurality of film layers to a punching step for generation of a plurality of microstructures, said punching being performed by use of a rigid stamp having a plurality of protrusions defining a plurality of cavities, wherein the depth of the cavities is larger than the thickness of the plurality of film layers, and lower than the combined thickness of the plurality of film layers and the deformable surface part of the handling substrate, thereby allowing the protrusions of the rigid stamp to penetrate all the way through the plurality of film layers during punching to divide the plurality of film layers into a plurality of separated microstructures.

258. The method according to claim 257, wherein the punching step comprises:
applying a pressure to the rigid stamp for a given time period, said pressure having a value being high enough to allow the protrusions of the rigid stamp to penetrate all the way through the plurality of film layers within the given time period.

259. The method according to claim 258, wherein a relatively high pressure is applied to the rigid stamp for said time period, said pressure having a value of not lower than 6 bar, such as not lower than 7 bar, such as not lower than 8 bar, such as not lower than 9 bar, or such as not lower than 10 bar.

260. The method according to claim 258, wherein said given time period is no longer than 3 minutes, such as no longer than 2 minutes, or such as no longer than 1 minute; wherein for at least a first or start part of said given time period, no external heat is supplied to the film layers;
wherein external heat is supplied to the film layers during a second or end part of said given time period; and
wherein the external heat is supplied to the film layers for a relatively short time period being no more than 30 seconds.

261. The method according to claim 258, wherein no external heat is supplied to the film layers during said given time period; wherein external heat is supplied to the film layers after expiry of said given time period; and
wherein external heat is supplied to the film layers after the expiry of said time period, said external heat being supplied for a relatively short time period being no more than 30 seconds.

262. The method according to claim 260, wherein the external heat is controlled for the film layers to reach a temperature not lower than 40° C.

263. The method according to claim 258, wherein a relatively low pressure is applied to the rigid stamp for said time period, said pressure having a value of not higher than 5 bar, such as not higher than 4 bar, such as not higher than 3 bar, or such as not higher than 2 bar; and wherein said given time period is no shorter than 5 minutes, such as no shorter than 10 minutes, or such as no shorter than 15 minutes.

264. The method according to claim 263, wherein external heat is supplied to the film layers during at least a part of said given time period, such as during at least half of said given time period, or such as during at least ¾ of said time period; and
wherein the supplied external heat is controlled for the film layers to reach a temperature not lower than 50° C., such as not lower than 65° C.

265. The method according to claim 250, wherein the thickness of each of the provided film layers is in the range of 5 to 200 μm; and
wherein the handling substrate holds a deformable surface layer having a thickness in the range of 5 to 200 μm.

266. The method according to claim 250, wherein the deformable surface or deformable surface layer of the handling substrate is subjected to an oxygen plasma treatment prior to depositing the one or more film layer(s).
